(19) United States
(12) Patent Application Publication
(10) Pub. No.: US 2022/0395614 A1
(43) Pub. Date: Dec. 15, 2022

(54) BIOMIMETIC POLYMERIC COMPOSITE FOR HEART VALVE REPAIR

(71) Applicant: THE TRUSTEES OF COLUMBIA UNIVERSITY IN THE CITY OF NEW YORK, New York, NY (US)

(72) Inventors: Mingze SUN, Englewood, NJ (US); David KALFA, Long Island City, NY (US)

(21) Appl. No.: 17/849,545

(22) Filed: Jun. 24, 2022

Related U.S. Application Data
(63) Continuation-in-part of application No. PCT/US2020/067002, filed on Dec. 24, 2020.
(60) Provisional application No. 62/953,768, filed on Dec. 26, 2019.
(60) Provisional application No. 62/976,252, filed on Feb. 13, 2020.
(60) Provisional application No. 63/310,688, filed on Feb. 16, 2022.

(51) Int. Cl. A61L 27/50; A61L 27/34; A61L 27/18; A61L 27/56

(52) U.S. Cl. CPC A61L 27/505; A61L 27/34; A61L 27/18; A61L 27/56; A61L 2400/18; A61L 2430/20; A61L 2400/02

(57) ABSTRACT

A biomimetic, polymeric composite biomaterial designed as a heart valve leaflet substitute that can be used for heart valve repair and/or to fabricate a new-generation of durable heart valve prosthesis.

### CROSS REFERENCE TO RELATED APPLICATIONS

This application is a Continuation-in-Part of International Application No. PCT/US2020/067002, filed Dec. 24, 2020, which claims priority to U.S. Provisional Application No. 62/953,768 filed Dec. 26, 2019 and U.S. Provisional Application No. 62/976,252 filed Feb. 13, 2020, the disclosures of which are incorporated by reference herein in their entirety. This application claims priority to U.S. Provisional Application No. 63/310,688, filed Feb. 16, 2022, the disclosure of which are incorporated by reference herein in their entirety.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### FIELD

A biomimetic, polymeric composite biomaterial designed as a heart valve leaflet substitute that can be used for heart valve repair and/or to fabricate a new-generation of durable heart valve prosthesis. In some embodiments, the polymeric composite biomaterial is in the form of a patch.

### BACKGROUND OF TECHNOLOGY

Valve replacement in adults and children has inherent problems associated with anticoagulation (mechanical valves) or durability (bioprosthetic heart valves), which leads to the failure of the prosthesis and increases the probability for reoperation and the accompanying risk. Thus, valve repair is always the preferred approach, compared to replacement.

Valve repairs frequently require the use of cardiovascular patches to perform leaflet augmentation or extension. Current available patches used for valve repair, such as bovine pericardium, porcine intestinal submucosa extracellular matrix, expanded polytetrafluoroethylene, fresh autologous pericardium and glutaraldehyde-treated autologous pericardium, all have intrinsic limitations and drawbacks that affect their long-term durability and mechanical performance, leading to structural degeneration (SD) of the patch and of the repaired valve leaflet.

Baird et al. used PhotoFix® patches in young patients for valve repair and showed cases of degeneration, calcification and inflammation. (Baird, C. W. et al. Photo-Oxidized Bovine Pericardium in Congenital Cardiac Surgery: Single-Centre Experience. Interact CardioVasc Thorac Surg 2017, 24 (2), 240-244). Hofmann et al. reported a high rate of mechanical failure of CorMatrix® patches for aortic valve repair leading to valve insufficiency. CardioCel® patch was also associated with a significant risk of patch failure and the need for reoperation in large series. (Hofmann, M. et al., Congenital Aortic Valve Repair Using CorMatrix®: A Histologic Evaluation. Xenotransplantation 2017, 24 (6), e12341). Pavy et al. summarized that the discrepancy between the mechanical property (the elasticity) of the patch and the native tissue was the result to cause severe aortic stenosis in infants and lead to patch failure. (Pavy, C. et al. Initial 2-Year Results of CardioCel® Patch Implantation in Children. Interactive CardioVascular and Thoracic Surgery 2018, 26 (3), 448-453), It is also in line with Tremblay's conclusion, which reported that the difference in the mechanical properties of aortic tissues and prosthetic material was a main factor to contribute the unwanted hemodynamic effects leading to patch failure. (Tremblay, D. et al. Comparison of Mechanical Properties of Materials Used in Aortic Arch Reconstruction. The Annals of Thoracic Surgery 2009, 88 (5), 1484-1491).

To overcome these drawbacks, implantable patches or grafts with native-like structures and tunable mechanical performance close to the ones of the native valve leaflets were attempted. In this regard, a polyvinyl alcohol (PVA)-bacterial cellulose (BC)-based hydrogel was designed to mimic the mechanical properties of the native valve leaflet. But, the degradable nature of PVA over time, poor design of the patch and lack of data on the durability of the composite hindered its application as a stable cardiac patch. Another composite fabrication involved the combination of poly(ethylene glycol) (PEG) hydrogel and polycaprolactone (PCL) fiber for heart valve tissue engineering, but this composite material demonstrated an anisotropic behavior on the unicycle tensile test only and had a linear stress-strain behavior that was different from the non-linear behavior of native leaflets. This may cause valvular interstitial cells (VICs) to experience greater stresses, impact VIC activation and extracellular matrix (ECM) remodeling, leading to calcification.

Masoumi et al. attempted a tri-layered scaffold designed to mimic structural and anisotropic mechanical characteristics of the native leaflet. (Masoumi, N. et al., A. Tri-Layered Elastomeric Scaffolds for Engineering Heart Valve Leaflets. Biomaterials 2014, 35 (27), 7774-7785). But this biodegradable patch degraded at a fast rate with a loss of mechanical strength from 3.02 MPa to 1.63 MPa in 4 weeks. In comparison, native valve aortic and pulmonary leaflets keep a stable modulus of, 3.84 and 2.55 MPa, respectively over time. Despite aiming at replicating the architecture of native leaflets, Masoumi's patch is not a mechanically stable option for clinical use. Thus, there remains a need for a stable, functional and biomimetic patch that overcomes the drawbacks of previous patches, including a clinical need for a new type of patch that can achieve a better durability after implantation in patients

### SUMMARY

In one aspect, a stable biomimetic polymeric biomaterial is provided. The biomaterial includes at least two layers including a Fibrosa-mimic (“F-mimic”) layer, a Spongiosa-mimic (“S-mimic”) layer, and a Ventricularis-mimic (“V-mimic”) layer. In some embodiments, the F and V layers are anisotropic and the S layer is a shock absorbing layer. In some embodiments, the F-mimic layer and the V-mimic layer are made of polycarbonate polyurethane (PCU) film, enhanced with aligned, electrospun polycaprolactone (PCL) fibers, and the S-mimic layer is made of PCU foam. In some embodiments, the stable biomimetic polymeric biomaterial includes two to five layers. The stable biomimetic polymeric biomaterial may be devoid of animal-derived tissue, thus, in some embodiments it has no animal-derived tissue. The biomaterial may be used to make a patch, such as for treating a heart defect, or a prosthetic heart valve.

In another aspect, a polymeric, biomimetic customized biomaterial patch (“BCP”) that replicates the structure-function driven architecture of native valve leaflets is provided and described herein. In one embodiment, the BCP replicates the three-layer architecture and the anisotropic mechanical properties of a native leaflet. Thus, in one embodiment, the BCP comprises a composite body including three polymeric layers. In this regard, the layers include a Fibrosa-mimic (“F-mimic”) layer; a Spongiosa-mimic (“S-mimic”) layer; and a Ventricularis-mimic (“V-mimic”) layer. In some embodiments, the F and V layers are anisotropic and the S layer is a shock absorbing layer. In some embodiments, the F-mimic layer and the V-mimic layer are made of polycarbonate polyurethane (PCU) film, enhanced with aligned, electrospun polycaprolactone (PCL) fiber mesh, and the S-mimic layer is made of PCU foam. In some embodiments, the biomimetic patch is entirely polymeric, i.e., lacks animal-derived tissue. The tri-layered patch can be modified and tuned to achieve the specific mechanical requirement, as well as have a low antigenicity and lower risk for structural valve degeneration.

The BCP described herein, as compared to three commercial patches, exhibits an anisotropic mechanical behavior and mechanical stiffness (6.20±1.83 MPa and 1.80±0.21 MPa in circumferential and radial directions, respectively), which is more similar to the native aortic valve leaflets than any currently available commercial patches. The BCPs also exhibits greater durability and greater biocompatibility. In vivo rat subcutaneous tests also confirmed the BCP exhibits mechanical biostability and superior resistance to inflammation and calcification, compared to the commercial patches. Thus, the BCP embodied herein provide a new clinical-grade biomaterial patch useful for heart valve extension, augmentation or replacement in children and adults.

In yet another aspect, a novel polymeric valved device, such as an implantable prosthetic heart valve is provided. The implantable prosthetic heart valve comprises the biomaterial described herein. The implantable prosthetic heart valve may be an aortic valve, mitral valve, or tricuspid valve.

In yet a further aspect, methods for repairing a heart defect with the BCP and methods for delivering an implantable heart valve to a subject in need thereof is described and embodied herein.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1A shows a cross sectional view of the architecture of native heart valve;

FIG. 1B shows a cross sectional view of a tri-layered biomimetic patch in accordance with one embodiment of the disclosed subject matter;

FIG. 1C shows a cross sectional view of the V-mimic layer with aligned fibers in accordance with the embodiment in FIG. 1B in accordance with the disclosed subject matter;

FIG. 1D shows a cross sectional view of an embodiment of the biomaterial having aligned PCL fibers in the F-mimic layer and V-mimic layer in accordance with the disclosed subject matter;

FIG. 1E shows a top view and side perspective of the biomaterial including an S-mimic foam layer and a plurality of aligned fibers in accordance with the disclosed subject matter;

FIG. 1F is a chart showing the tensile properties of the biomaterial of FIG. 1E compared to native leaflets in accordance with the disclosed subject matter;

FIGS. 2A-2G show comparative mechanical properties of native leaflets tissue and commercial cardiac patch representatives in accordance with the disclosed subject matter;

FIGS. 3A-3I show structures and mechanical behaviors of electrospun fibers, fiber-enhanced layers, S-mimic layers and composite patches in accordance with the disclosed subject matter;

FIG. 4A shows speckled specimen was glued and embedded between the plates in accordance with the disclosed subject matter;

FIG. 4B shows schematic of pressure loading regimen in accordance with the disclosed subject matter;

FIG. 4C shows definition of the specimen coordinate system for tissue samples in accordance with the disclosed subject matter;

FIG. 4D shows results of cyclic loading for representative specimen in accordance with the disclosed subject matter;

FIGS. 4E-4G show contours of displacement components in X, Y, and Z directions at the maximum pressure in accordance with the disclosed subject matter;

FIG. 4H show results of the representative cycle of HAV deformation in accordance with the disclosed subject matter;

FIG. 5A shows an example of suture retention strength and thickness-normalized suture retention strength in accordance with the disclosed subject matter;

FIG. 5B is a schematic representation of a specimen during suture retention strength test;

FIG. 5C shows representative SRS curves of the commercial patches, the PCU films and the BCPs in accordance with the disclosed subject matter;

FIG. 5D shows the SRS difference found among the commercial patches, the PCU films and the BCPs in accordance with the disclosed subject matter;

FIG. 5E shows representative TN-SRS curves of the commercial patches, the PCU films and the BCPs in accordance with the disclosed subject matter;

FIG. 5F shows the TN-SRS difference found among the commercial patches, the PCU films and the BCPs in accordance with the disclosed subject matter;

FIG. 6A-6E show comparative biostability performance of three commercial patches, the PCU films/foams and BCPs in accordance with the disclosed subject matter;

FIGS. 7A and 7B shows biocompatibility performance: BSA protein adsorption and Ca2+ adhesion of commercial patches, the PCU film and the BCP in accordance with the disclosed subject matter;

FIG. 8A-8F show histological characterization (H&E, Alizarin Red), mechanical property and calcium quantification of the PCU film, Gore-Tex® patch and CardioCel® Patch after in vivo implantation in accordance with the disclosed subject matter; and

FIG. 9 is a graph showing commercial patches have a general stiffer performance than native tissues and BCPs. The flexural modulus, calculated from the bulge tests, displays a trend in accordance with the tensile modulus from the tensile tests, especially the one obtained in C/H direction (orange line vs red bar) in accordance with the disclosed subject matter.

FIG. 10(A) is a schematic drawing for the design of biomimetic, multilayered material (BMM) in accordance with exemplary embodiments of the disclosed subject matter.

FIG. 10(B) is a SEM image of fiber morphologies of aligned fibers.

FIG. 10(C) is a SEM image of random fibers

FIG. 10(D) is a SEM image of the cross-section of the F/V-mimic layer, the white arrows and the enlarged image display the PCL fibers embedded in the PCU film.

FIG. 10(E) is a cross-section of the S-mimic layer is displayed and shown its porous structure

FIG. 10(F) illustrates the tri-layer structure of Film-Foam-Film in accordance with the disclosed subject matter.

FIG. 11 illustrates the set-up for the flexural bulge test.

FIG. 12 illustrates the scheme of pressure loading regimen.

FIG. 13 illustrates a plot of the total load-unload cycle.

FIG. 14(A) illustrate representative stress-strain curves of Random PCL fibers (black), Aligned PCL fibers (red) and Fiber-enhanced Film (green).

FIG. 14(B) illustrate representative stress-strain curves of PCU Film (upper curve) and Foam (lower curve)), which are fabricated from the same concentration of PCU solution.

FIG. 14(C) illustrates stress-strain average curves of the disclosed subject matter, HAV and three commercial patches: Gore-Tex®, CorMatrix® and CardioCel® along circumferential direction (C-Direction).

FIG. 14(D) illustrates stress-strain average curves of the disclosed subject matter, HAV and three commercial patches: Gore-Tex®, CorMatrix® and CardioCel® along radial direction (R-Direction).

FIG. 14(E) illustrates the tensile modulus of all samples at strain level=15% and 40%. *P<0.05, **P<0.01 and ****P<0.0001 indicate significant difference between commercial patches and HAV.

FIG. 15(A) illustrates representative Stress-strain curves of the disclosed subject matter, HAV and three commercial patches: Gore-Tex®, CorMatrix® and CardioCel®.

FIG. 15(B) is an enlarged image of the black dotted rectangular area of FIG. 15(A) showing the curves in the range of stress=0-1 MPa.

FIG. 16 illustrates the tensile module for the BM (the disclosed subject matter) and other materials.

FIG. 17(A) illustrates the Suture Retention Strength (SRS) curves of the commercial patches, the PCU films and BMM (the disclosed subject matter).

FIG. 17(B) illustrates the SRS of BMM (the disclosed subject matter), PCU film and each commercial patch. The difference was found among the commercial patches (*p<0.05 for Gore-Tex® vs CardioCel® and **p<0.01 for CardioCel® vs CorMatrix®) (B).

FIGS. 17(C)-(D) illustrates representative Thickness-Normalized (TN)-SRS curves of the commercial patches, the PCU films and the disclosed subject matter. The TN-SRS of the disclosed subject matter had no significant difference compared to the commercial options, while PCU film has a significant higher TN-SRS compared to the rest of samples (**p<0.01 for PCU film vs the remaining groups).

FIG. 18 illustrates representative toughness calculations for BMM (the disclosed subject matter) and other materials.

FIG. 19(A) illustrates the tensile modulus change of all samples in 30 days in the accelerated oxidization solution.

FIG. 19(B) illustrates the tensile modulus of BMM (of the disclosed subject matter) in 30 days showed a stable mechanical performance.

FIG. 19(C) illustrates SEM images that show details on the surface morphology of BMM (of the disclosed subject matter) from 0-30 days.

FIG. 19(D) illustrates the BSA protein adsorption of the BMM (of the disclosed subject matter) and commercial patches.

FIG. 19(E) illustrates the Ca deposition of the PCU film, BMM (of the disclosed subject matter) and commercial patches.

FIGS. 20(A1)-(A9) and 20(B1)-(B6) illustrate histological characterization, mechanical property and calcium quantification of the PCU film, Gore-Tex® patch and CardioCel® Patch after in vivo implantation.

FIG. 20(C) illustrates calcium quantification data of three samples.

FIG. 20(D) illustrates the mechanical property of PCU film before and after implantation (D). P values of <0.01 (**) and <0.001 (***) were considered statistically significant.

FIG. 21 illustrates representative stress-strain curves for various materials.

FIG. 22 illustrates a polymeric heart valve prosthesis fabricated via suturing the BMM along the 3D-printed valve struts.

FIG. 23(A)-(C) illustrates the water contact angles for the BMM and several additional materials.

FIG. 23(D) illustrates the water contact angles for the BMM (the disclosed subject matter) and for several materials.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

In one aspect, a biomimetic polymeric biomaterial is provided. The biomimetic polymeric biomaterial is useful as a heart valve leaflet substitute and/or to fabricate a prosthesis. In one embodiment, the polymeric biomaterial is used to make a biomimetic customized biomaterial patch or BCP. In other embodiments, the biomimetic polymeric biomaterial is used to fabricate a polymeric valve prosthetic device. Thus, the biomimetic polymeric biomaterial may be used for treating a subject in need of heart valve repair and/or heart valve replacement.

Generally, the BCP comprises a body having a multi-layered polymeric composite biomaterial. For example but not limitation, the multi-layered polymeric composite biomaterial may include two to five layers. In one embodiment, the biomaterial is a tri-layered polymer composite. In this embodiment, the BCP is designed to mimic the architecture, i.e., three distinct tissue layers that compose the valve leaflets, and the mechanical properties of native leaflet tissue.

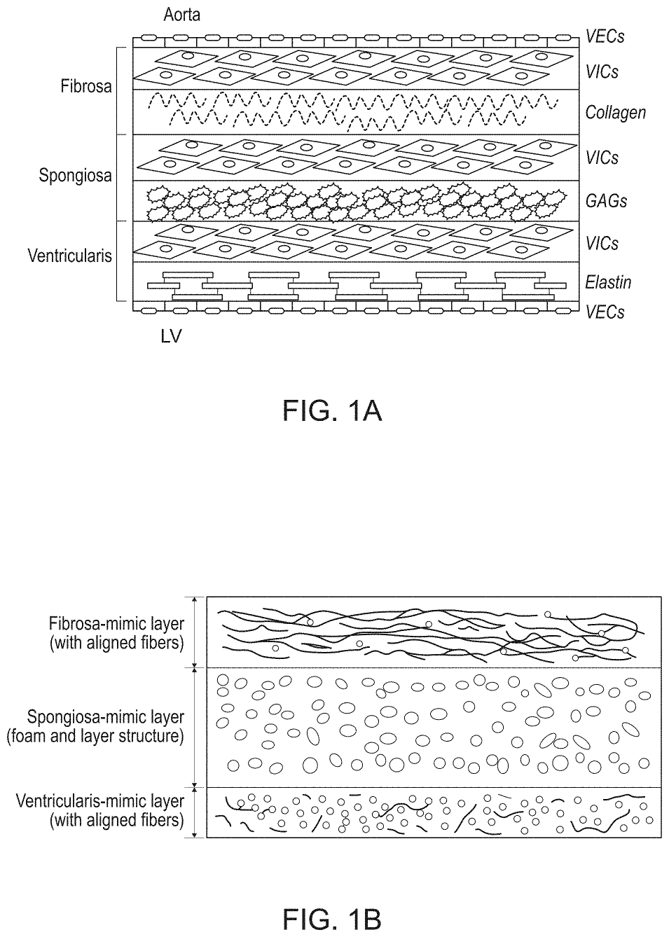

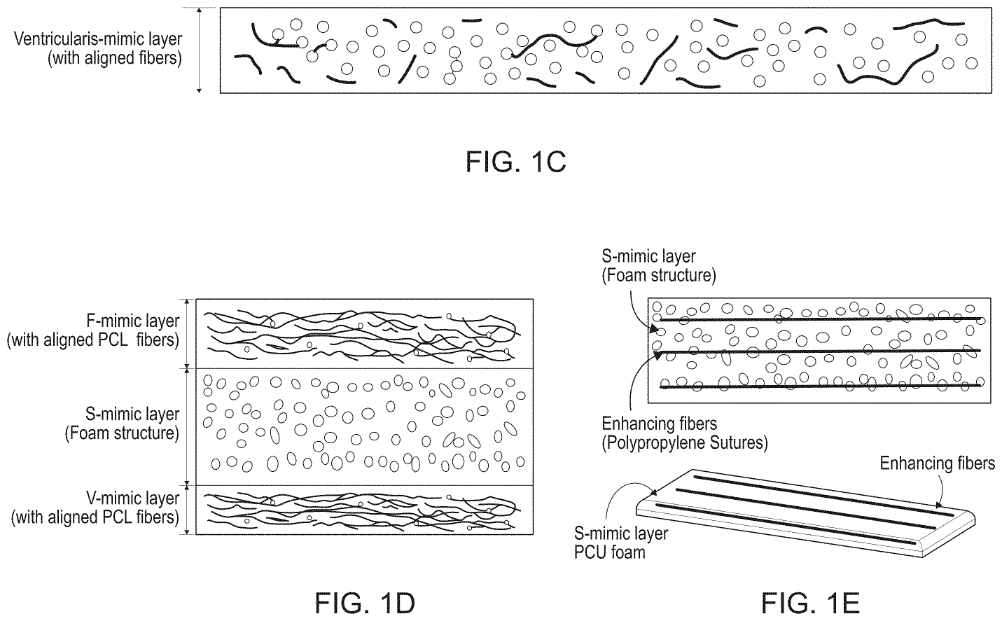

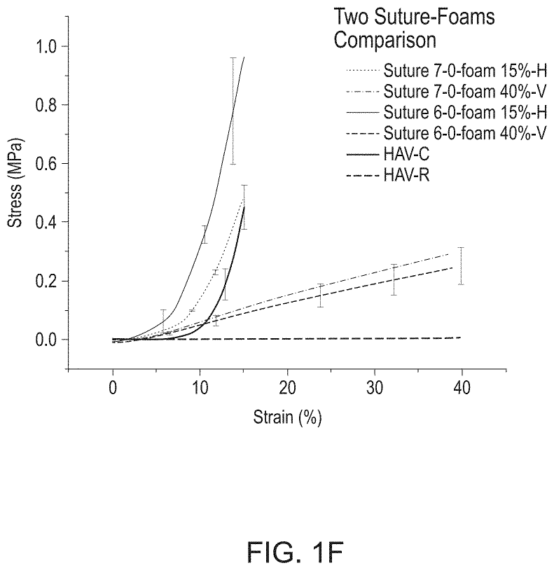

Referring to FIG. 1A, the architecture of the native heart valve 1000 is shown. The native heart valve tissue has a highly specialized architecture with three specific layers: the Fibrosa 1001, Spongiosa 1002, and Ventricularis 1003. They are composed of collagen, elastin and glycosaminoglycans (GAGs). The Fibrosa 1001 consists mainly of a dense network of corrugated type-I collagen fibers arranged in the circumferential direction, which provides the primary load-bearing properties of the heart valve. The Spongiosa 1002 is composed of highly hydrated GAGs and proteoglycans (PGs) as well as loosely arranged collagen and elastin. It acts as a cushion, enabling shear between the two other layers during loading and unloading, and absorbing the load resulting in minimal stress on the leaflet itself. The Ventricularis 1003 is comprised of less organized collagen fibers and radially oriented elastin sheets. It helps reduce large radial strains during the high blood flow over the valves when they are fully opened. The complex, highly organized structure of the valves leads to specialized mechanical properties necessary to withstand high trans-valvular pressures and low flexural stiffness.

Referring to FIG. 1B, the BCP 100 is illustrated. The BCP comprises a Fibrosa-mimic (“F-mimic”) layer 101, the Spongiosa-mimic (“S-mimic”) layer 102 and the Ventricularis-mimic (“V-mimic”) layer 103. The F-mimic layer 101 and V-mimic layer 102 are fiber-enhanced layers, comprising aligned PCL fibers and PCU film. The PCL fibers are embedded in the PCU matrix and dried as a fibrous film composite. The S-mimic layer 103 is a PCU foam layer that replicates the load-bearing mechanical role played by the native spongiosa. The F and V layers (101, 102) are anisotropic and mimic the mechanical properties of the native fibrosa 1001 and ventricularis 1002. The S layer 102 (foam) acts as a shock absorbing layer and has the same mechanical properties as the native spongiosa 1003. Thus, the BCP 100 replicates the heart valve leaflets' complex structure 1000. Referring to FIG. 1C, the V-mimic layer includes aligned fibers having a different direction as the fibers in the F-mimic layer, thus, the alignment of the fibers in these layers mimic that of native tissue. In another embodiment, referring to FIG. 1D, the F mimic layer and V-mimic layers may have PCL fibers aligned in substantially the same direction.

Referring to FIG. 1E, in another embodiment, the biomaterial is a composite structure including the S-mimic foam layer and a plurality of polypropylene fibers embedded in the foam structure to form a composite biomaterial. The polypropylene fibers, each have a longitudinal body and when embedded in the foam S-mimic layer are spaced apart from each other at a distance of between about 1 and about 3 mm. As one of ordinary skill in the art may appreciate, the length of the polypropylene fiber depends on the application and in particular the size of the biomaterial or patch desired. For example, patches that have a length of about 9 cm will include polypropylene fibers having a length of about 9 cm or less. The plurality of polypropylene fibers include 2 to 4 fibers. The fibers can also have different sizes. For example but not limitation, the diameter range for the fibers may be between about 0.030 to about 0.100 mm. In one embodiment, for example, the polypropylene fibers are monofilament sutures having sizes of 6-0, 7-0 and/or 8-0 (USP designation).

It has been found that the biomaterial comprising the S-mimic foam layer and a plurality of polypropylene fibers embedded in the foam structure to form a composite biomaterial offers mechanical properties substantially the same as native leaflet tissue, as shown in Table 1A below. As shown, the biomaterial in some embodiments exhibits a tensile modulus in a C/H direction of about 8 to about 16 MPa.

TABLE 1A
Tensile Properties of Modified Version of BCPs and Native Leaflets

| | Tensile | Strain (%) | | |
|---|---|---|---|---|
| | Modulus | where to obtain | Tensile Modulus | Strain (%) |
| | (MPa) | the tensile | (MPa) | where to obtain the |
| Sample | C/H Direction | modulus | R/V Direction | tensile modulus |
| Human Aortic Valve | 16.34 ± 0.42 | 14%-15% | 0.03 ± 0.01 | 39%-40% |
| Leaflet (HAV) | | | | |
| Suture 6-0 foam | 15.27 ± 0.05 | 14%-15% | 0.58 ± 0.005 | 39%-40% |
| composite | | | | |
| Suture 7-0 foam | 7.84 ± 0.15 | 14%-15% | 0.67 ± 0.002 | 39%-40% |
| composite | | | | |

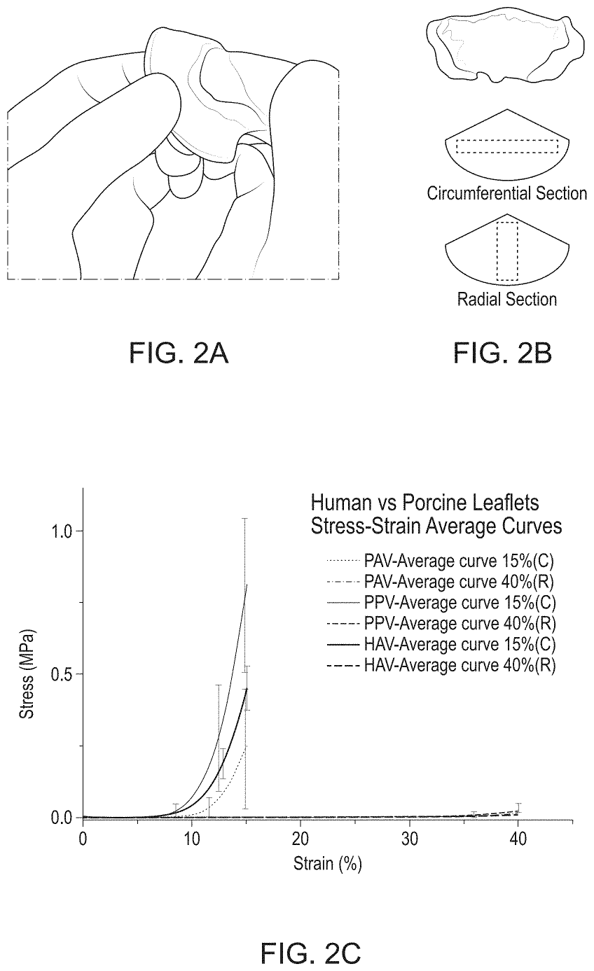

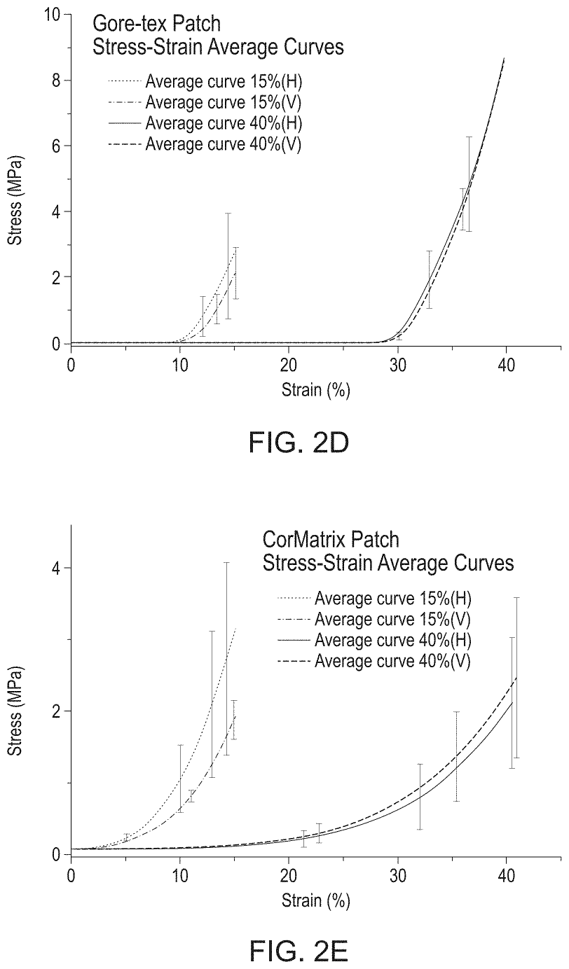

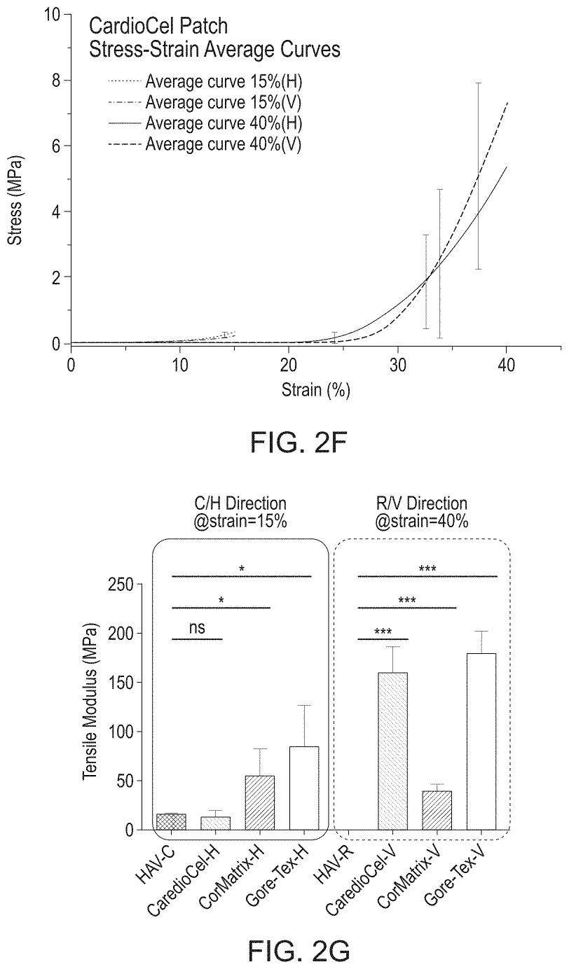

Referring to FIG. 1F, various embodiments of the biomaterial are compared in a polypropylene suture—PCU foam composite to native leaflet tissue. As shown, the biomaterial may include polypropylene suture 7-0-foam 15%-Horizontal (H), polypropylene suture 7-0-foam 40%-Vertical (V), polypropylene suture 6-0-foam 15%-H and polypropylene suture 6-0-foam 40%-V. For example, a first embodiment is a 6-0 suture-PCU foam composite and a second embodiment is a 7-0 suture-PCU foam composite. Three samples shown in Table 1A, (2 suture-foams and 1 native tissue) were tested from two directions, i.e., the H/C direction and V/R direction. As used herein, H is the horizontal direction of a 2D biomimetic patch; it is used to represent similar direction, circumferential direction of 3D native leaflets. So, H=C. Similarly, V=R. Referring back to FIG. 1F, a comparison of the solid curves representing the suture 7-0 foam composite, suture 6-0 foam composite and HAV in the H/C direction, they are relatively close. Additionally, the dash curves representing suture 7-0 foam composite, suture 6-0 foam composite and HAV in the H/C direction in the V/R direction, are also close. These data indicate that the embodiments of the suture foam composite are much more similar to native tissue than other commercial patches (FIG. 2G).

Exemplary Materials and Method for fabricating embodiment of FIG. 1D. Carbothane™ AC-4075A, Polycarbonated-based polyurethane (PCU) was ordered from Lubrizol. Dimethylacetamide (DMAC) was purchased from Acros organics and used as the solvent to dissolve PCU. Polycaprolactone (PCL, Mw=80,000) was purchased from Sigma-Aldrich. Chloroform and methanol with 3:1 molar ratio, was used to dissolve PCL and prepared as the electrospinning solution. Commercially available patches including Gore-Tex® (W. L. Gore and Associates, Flagstaff, Ariz., USA), CorMatrix® (Cardiovascular, Inc, Atlanta, Ga., USA) and CardioCel® (Admedus, Toowong, Queensland, Australia), porcine heart valves (obtained from a local slaughterhouse), and the CryoValve® aortic human valve (CryoLife Inc., Kennesaw, Ga., USA) were used as controls. Leaflets from porcine valves and human homograft were dissected and kept intact in PBS.

The BCP 100 was prepared by a combination of three native-tissue mimicking layers, respectively named the Fibrosa-mimic 101 (F-mimic) layer, the Spongiosa-mimic (S-mimic) layer 102 and the Ventricularis-mimic (V-mimic) layer 103. The F-mimic layer and V-mimic layer were designed as fiber-enhanced layers, composed of aligned PCL fibers and PCU film in order to replicate the anisotropy of these layers. The S-mimic layer was designed as a PCU foam to replicate the load-bearing mechanical role played by the native spongiosa. The structure of this BCP is shown in FIG. 1D. As shown, FIG. 1D has aligned PCL fibers in both the F-mimic layer and the V-mimic layer. As shown, fibers are in the same direction in the F and V-mimic layers.

Fabrication of the F-mimic 101 and the V-mimic 102 fiber enhanced layers. Using a 15% PCL solution prepared in a mixed solvent (Chloroform:Methanol=3:1), PCL fibers were produced by electrospinning with the following parameters: a flow of 1 ml/hour, a voltage of 20 kV voltage and a distance of 15 cm between the nozzle and drum collector. The solution was spun towards a rotating collector at a rate of 1600 rpm to collect the aligned fibers. The fibers were allowed to dry overnight in a chemical hood for solvent evaporation before the following fabrication and characterization. The collected, aligned PCL fibers were embedded in solution-casted PCU film. The 15% PCU solution was casted by a doctor-blade coater through a 500 μm gap to control the film thickness. The fiber-solution composite was cured overnight in a chemical hood to evaporate the solvent and form the fiber-enhanced layers.

Fabrication of the S-mimic layer 103. To produce the S-mimic layer, the 15% PCU solution was casted by a doctor-blade coater to create a film with a fixed thickness of 1500 μm. Subsequently, the film was immersed in deionized water for 24 hours. Then, the solvent-exchanged PCU film was frozen under −80° C. Lyophilization was conducted on the frozen PCU film at 0.1 mBar, −40° C. for 72 hours and turned into a porous layer to work as the S-mimic layer.

Fabrication of the BCP 100. The F-mimic layer was casted to form the fiber-enhanced layer. After 1 hour drying in the hood, the S-mimic layer was put over the casted composite and dried with the fiber-enhanced layer together in the chemical hood. Then this two-layer composite was put over the V-mimic layer to fabricate the BCPs.

Morphology Characterization. To characterize the PCL aligned fibers, each mimic layer and the BCPs, the specimens were sputter coated with gold/platinum and imaged with a Zeiss Sigma VP scanning electron microscope (SEM) at an accelerating voltage of 3 kV. SEM images were used for the visual inspection of fiber's orientation, mimic layer and BCPs' inner structures and their surface quality.

Tensile mechanical testing. The mechanical properties were measured with an Instron 5848 mechanical tester with a 50 N load cell at a strain rate of 10% s-1. The specimens were cut as 5 mm×20 mm stripes (for non-tissue samples) or 3 mm×10 mm ones (for the native tissue samples) in two different directions, horizontally/circumferentially (H or C direction) and vertically/radially (V or R direction), shown in FIG. 2B. The thickness of the specimens was measured at three different points with a digital caliper (Mitutoyo America Corp, Aurora, Ill., USA) and the values were averaged. Four to six specimens for each samples were repeatedly stretched for 20 cycles, either to a maximal strain of 15% in H/C direction or to a maximal strain of 40% in V/R direction. Missirlis and Chong, Brewer et al. and Thubrikar et al. have all reported in vivo AV leaflet strains to be approximately 10-15% and 30-40% in the circumferential and radial directions, from systole to diastole respectively. After the first 5 preconditioning cycles, the subsequent 15 cycles of stress-strain curves were recorded and averaged and the tensile modulus E were calculated as the Equation 1 below:

$$E = \frac{Δ σ}{Δ ε} \qquad [ 1 ]$$

where

$$Δ σ = \frac{Δ F}{{w}_{0} {T}_{0}} and Δε = \frac{Δ l}{{l}_{0}}$$

are engineering stress and engineering strain. l₀, w₀, and T₀ are the dimension (length, width and thickness) of the specimen, Δl is the change of elongation in length, and ΔF is the change of the force. Then the average curves and the tensile modulus at the strain of 15% or 40% were used to compare the mechanical performance in different directions and to assess anisotropy. Then the average curves and the tensile modulus at the strain of 15% or 40% were used to compare the mechanical performance in different directions and to assess anisotropy.

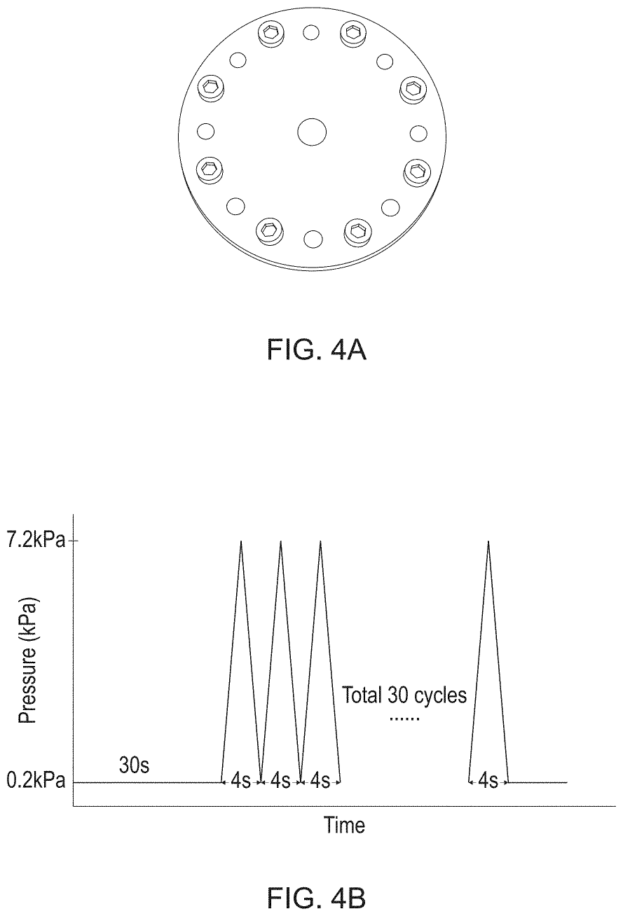

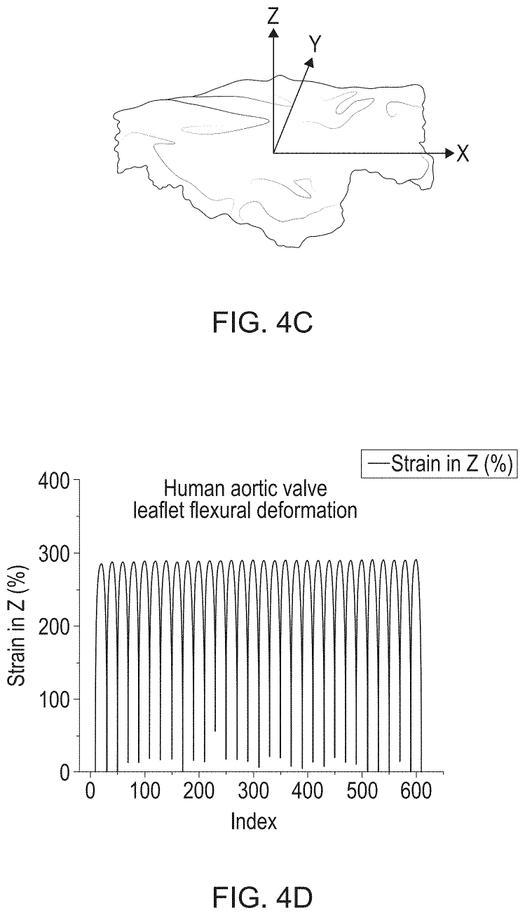

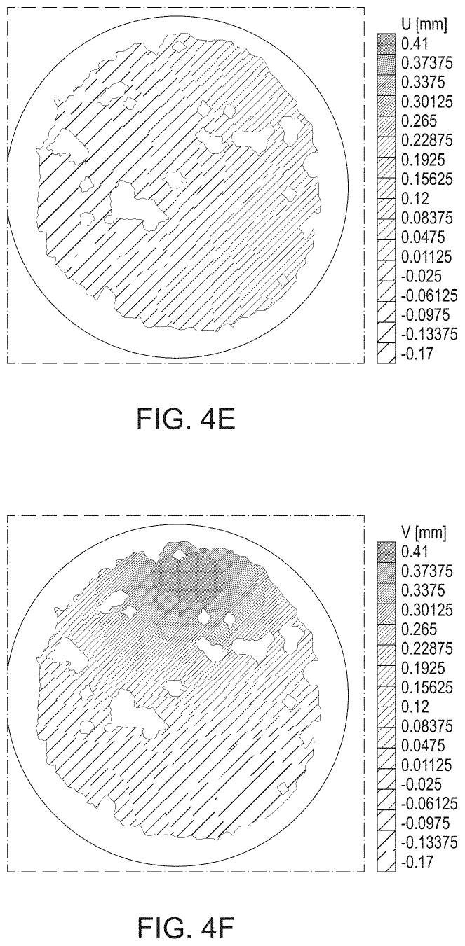

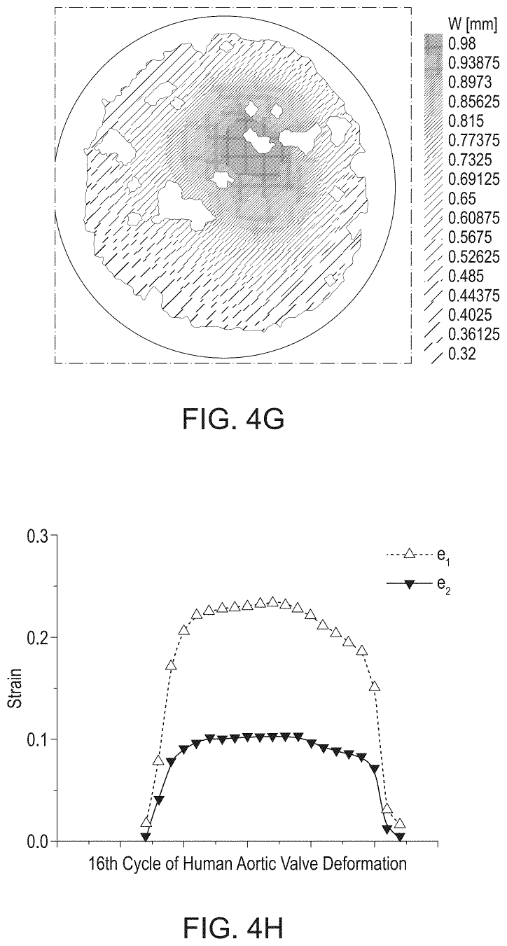

Flexural mechanical testing. Flexural properties of the commercial patches, leaflet tissues and BCPs were tested via the bulge tests. All samples were pre-cut as the circular planar specimens using fine dissectors. Thickness was evaluated by averaging three measurements taken at specimen's center with a digital caliper. The diameter of the caliper's contact plate was 10 mm, which was larger than the circular test area with a diameter of 6 mm; thus the specimens were assumed to be uniform in thickness. The specimens were speckled with black India ink to allow for DIC deformation tracking. The specimens were then glued between two plates with holes of 6 mm diameter (FIG. 4A). The embedded specimen was secured onto a custom inflation chamber through the holder.

The specimens were inflated by a custom-made displacement-driven syringe injection of PBS into the custom-made pressurization chamber. The pressure was monitored by a pressure transducer with 0-8 kPa range. The loading regimen was programmed using LabView (V2020, National Instruments, Austin, Tex.) and displayed in FIG. 4B. The specimen was brought to a baseline pressure of 0.2 kPa and held for 30 seconds prior to cyclic testing to ensure the specimen was at equilibrium. The specimens were subjected to 30 load-unload cycles at a rate of 3.5 kPa/s from the baseline pressure to a maximum pressure of 7.2 kPa. These cycles were used to mimic the deformation under the quasi-physiological pressure level.

The deforming specimen surface was imaged by two stereoscopically arranged cameras with 20 mm focus lengths at an aperture of f/4. The optical axes of the cameras were positioned 35 cm above the chamber and fixed with a total angle of 12°. This configuration had a depth of field in front over 1.5 cm, sufficient to capture the deformation of the specimen between 0.2-7.2 kPa. Images were collected during testing at a rate of 10 Hz by VicSnap 2009 and correlated by Vic3D (V8, Correlated Solution, Inc. Columbia, S.C., USA).

To calculate the pressure and displacement resultants, the measured pressure and displacement were tared by the baselines, resulting in relatively zero stress and strain at the reference state. The method of calculating the flexural modulus Eflex is provided below. The sample in this test was modeled as a circular thin plate with edges fully fixed. The pressure was evenly distributed on the bottom surface of the sample. The governing equation and boundary conditions of this case could be expressed in cylindrical coordinates (r, ø, z) as

$${∇}^{2} {∇}^{2} w = - \frac{Δ P}{D} s . t \frac{w ( R ) = 0}{ϕ ( R ) = 0} \qquad [ 2 ]$$

where w is the displacement of z direction (defined as the out-of-plane direction) at a point of the thin plate, R is the radius of the plate, and ΔP is the pressure exerted. D is the flexural rigidity defined as

$$\frac{{E}_{flex} {T}_{0}^{3}}{12 ( 1 - {v}^{2} )}$$

T0 is the thickness of the specimen. The solution to this equation is

$$w ( r ) = - \frac{Δ P}{64 D} ( {R}^{2} - {r}^{2} ) \qquad [ 3 ]$$

At the center point (r=0), the flexural modulus could be expressed as

$${E}_{flex} = Δ P \frac{3 {R}^{4} ( 1 - {v}^{2} )}{16 {T}_{0}^{3} Δ W} \qquad [ 4 ]$$

where ΔW is the change of the displacement in z-direction. Here, all the materials were assumed to be incompressible, so the Poisson's ratios were all set as 0.5.

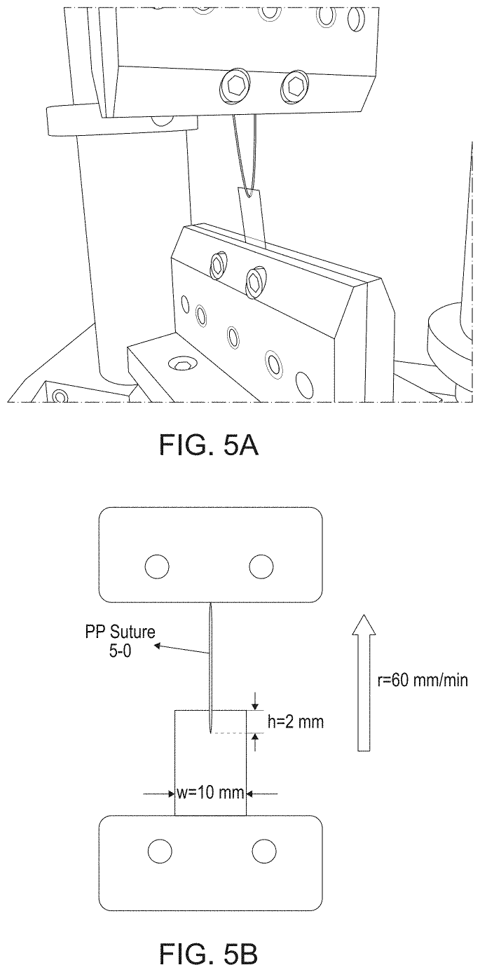

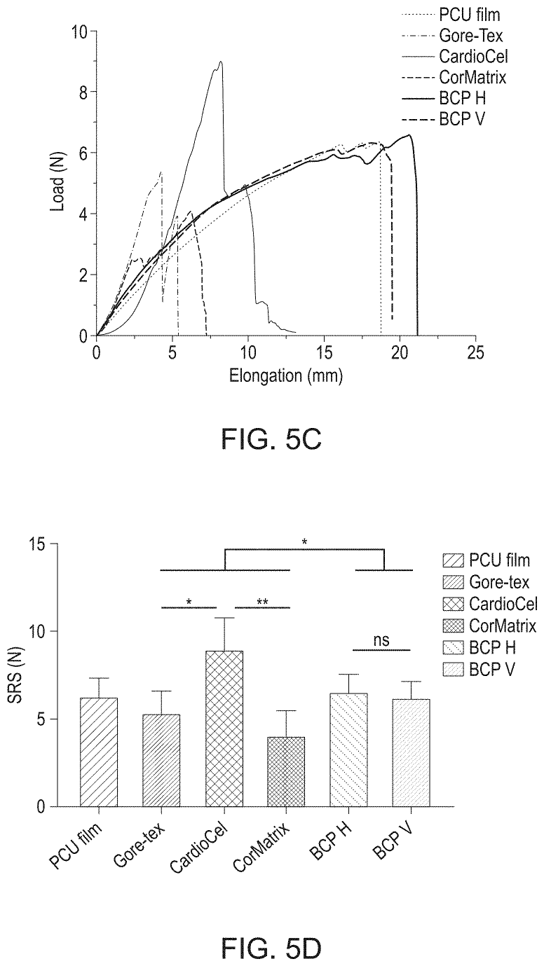

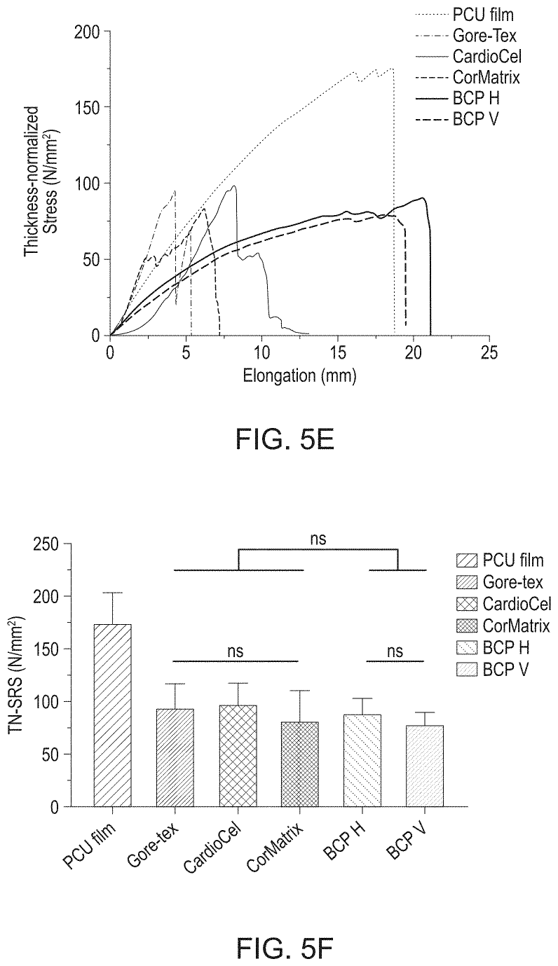

Suture retention testing. The suture retention capabilities of the three commercial patches, PCU films and the BCPs were tested following the steps described in Pensalfini et al.'s work, using Instron 5848 tensile machine (FIG. 5A). Prolene 5-0 suture was inserted 2 mm from the end of the 10×15 mm specimen and through the specimen to form a half loop. The suture was pulled at the rate of 50 mm/min crosshead speed (FIG. 5B). Five specimens were tested in each group. The force (N) required to pull the suture through and/or cause the specimen to fail was recorded as the suture retention strength (SRS). A thickness normalized suture retention strength (TN-SRS, N/mm²) was calculated, by dividing the suture retention strength by the area of the sample over which the load was applied:

$$TN - SRS = \frac{SRS}{Suture Thread Diameter + Sample Thickness} \qquad 5 ]$$

and compared among all the samples.

Biostability testing. Specimens of the commercial patches, PCU films/foams and BCPs were pre-cut as 5 mm×30 mm and submerged into 2 mL vials filled with an in vitro solution of 20% hydrogen peroxide (H2O2)/0.1M cobalt chloride (CoCl2). The in vitro solution was refreshed twice a week, and all testing were done at 37° C. After a period of 5, 10, 14, 15, 20, 24, and 30 days, the specimens were removed, rinsed thoroughly in deionized-water, dried in the hood, then cut into two parts (5 mm×25 mm and 5 mm×5 mm). The former was tested via the tensile mechanical testing and the tensile modulus at strain=15% was calculated. The latter was analyzed by SEM to inspect the surface quality.

Biocompatibility testing. Bovine Serum Albumin (BSA) static protein-adsorption experiments. For static protein-adsorption tests, 1 mg mL-1 BSA solution was prepared in PBS (pH 7.4). Commercial patches and PCU films were cut into specimens (50 mm×10 mm) and immersed in 10 mL 1 mg mL-1 BSA solution in a test tube. BSA adsorption was conducted under vibration at 37° C. for 3 hours to allow for adsorption equilibrium. Then the specimens were rinsed with PBS, the remaining proteins adsorbed on the surfaces were removed with a 1 wt % aqueous solution of sodium dodecylsulfate (SDS), similar to the work done by Song et al. The experiments were performed with five measurements for each specimen. BSA content was measured using a NanoDrop™ spectrophotometer at a wavelength of 280 nm and then the amount of adsorbed BSA on specimens was calculated.

Calcium-ion (Ca2+) adhesion experiments. The Ca2+ adhesion experiments were performed in a metastable calcium phosphate (MCP) solution. The purpose of using this MCP solution is to obtain calcium-phosphate compounds which can precipitate out from the solution and deposit on the tested specimens, in order to test the samples' calcification resistance in in vitro studies. Similar experiments were performed as reported earlier. In brief, 3.87 millimole (mM) CaCl₂), 2.32 mM K₂HPO₄ and 0.05M Tris buffer were solved in 1000 ml of de-ionized water, to yield a Ca/PO₄ ratio of 1.67.

This solution is more physiologically representative of hydroxyapatite, with a Ca/PO₄ ratio of 1.67, which is the most common form of calcium minerals in the vascular calcification process. Commercial patches and PCU films were cut into specimens (5 mm×30 mm) and immersed in 2 mL MCP solution individually. This experiment was conducted under vibration at 37° C. and solution was changed every 48 hours to ensure an adequate ion concentration. The specimens were removed after 16 days and rinsed with water to remove excess solution and loosely attached deposits. The specimens were dried in the vacuum oven at 70° C. overnight, accurately weighed, and hydrolyzed in 2 mL of 2 N HCl for 24 hours at 50° C. The calcium concentration was determined from HCl hydrolysate, using calcium colorimetric assay.

Rat subcutaneous implant model. In accordance with NIH guidelines for the care and use of laboratory animals (NIH Publication #85-23 Rev. 1985), all animal protocols were approved by the Institutional Animal Care and Use Committee (IACUC) of Columbia University (Protocol #AC-AABD5614).

Eighteen specimens (diameter=8 mm) of PCU film (n=6), Gore-Tex® patch (n=6) and CardioCel® patch (n=6) were implanted in the subcutaneous position of three rats. Following induction of anesthesia, fur clipping, and standard sterile prepping and draping, six subcutaneous pockets were created on the dorsal surface of each rat. One specimen was implanted into each pocket, after which all wounds were re-approximated with surgical clips. The rats were sacrificed at 8 weeks with an overdose of isoflurane (Euthenase).

Histology. The implanted specimen was retrieved while still contained in host tissue, fixed in 10% neutral buffered formalin and processed using paraffin-embedding techniques. Slides were stained with Hematoxylin and Eosin and Alizarin Red stains. In each specimen, both the patch and the surrounding host tissue were evaluated.

Calcium Content & mechanical test. Samples were analyzed for calcium content using calcium colorimetric assay as described in Calcium-ion adhesion experiments described above. Briefly, the specimen disks were removed from host tissue, fixed in formalin and solvent-exchanged in DI-water. Following with the lyophilization, the net weight of the specimen disks were acquired. After hydrolyzing in nitric acid, the calcium content was quantitated. Results are reported as microgram calcium per milligram dry specimen weight. The PCU disks can be separated from the host tissue after lyophilization. This specimen's mechanical performance was also evaluated as described in section 2.4 and its tensile modulus at the strain=15% was recorded, to compare with the control, unplanted sample.

Statistical Analysis. Statistical analyses of the tensile mechanical properties, biostability mechanical tests, protein adsorption and calcium adhesion tests were performed using one-way analysis of variance (ANOVA). P values less than 0.05 were considered statistically significant (*P<0.05, **P<0.01, ***P<0.001 and ****P<0.0001). And differences between samples within the groups were evaluated using a student's t-test, or Tukey's multiple comparisons test followed by ANOVA. GraphPad Prism 7 (San Diego, Calif., USA), statistics package, was used to obtain statistical significance for the study above.

Results. Structure and Mechanical Properties. Tensile properties of native tissues and commercial patches. We performed cyclic, uniaxial tensile tests on native leaflets and commercial patches in order to compare the mechanical performance of our BCP with these reference tissues (FIG. 2). For native leaflets samples, after the first 5 pre conditioning tensile cycles, the average of the up-curves from the subsequent 15 cycles exhibit a residue elongation and then an increase in the slope of the stress-strain curves which is attributed to the deformation and stretch of fiber networks in the tissue. This increase is accentuated in the circumferential direction (C-direction) compared to the radial direction (R-direction) due to the existence of oriented, crimped collagen fibers in circumferential direction (FIG. 2C). The tensile modulus was calculated using the equation 1 at the strain of 15% and 40%, for the C-direction and R-direction respectively. It shows that the human aortic valve leaflets (HAVs) have a higher tensile modulus value, 16.34±0.42 MPa, than the value of porcine aortic valve leaflets (PAVs), 8.71±9.88 MPa, at the strain=15% in C-direction. For same species, porcine pulmonary valve leaflets (PPVs) are much stiffer than PAVs, 20.00±13.41 MPa vs 8.71±9.88 MPa in C-direction and 0.52±0.85 MPa vs 0.19±0.20 MPa in R-direction (Table. 1 and FIG. 2C). All these human and porcine leaflets display a highly anisotropic performance and are much stiffer in the C-direction than the R-direction.

TABLE 1B
Tensile Properties of Native Tissues and Commercial Patches

| | | Strain (%) | | |
|---|---|---|---|---|
| | Tensile Modulus | where to obtain | Tensile Modulus | Strain (%) |
| | (MPa) | the tensile | (MPa) | where to obtain the |
| Sample | C/H Direction | modulus | R/V Direction | tensile modulus |
| Human Aortic Valve | 16.34 ± 0.42 | 14%-15% | 0.03 ± 0.01 | 39%-40% |
| Leaflet | | | | |
| Porcine Aortic Valve | 8.71 ± 9.88 | 14%-15% | 0.19 ± 0.20 | 39%-40% |
| Leaflet | | | | |
| Porcine Pulmonary | 20.00 ± 13.41 | 14%-15% | 0.52 ± 0.85 | 39%-40% |
| Valve Leaflet | | | | |
| Gore-Tex ® Patch-(H) | 85.04 ± 41.27 | 14%-15% | 181.85 ± 55.50 | 39%-40% |
| Gore-Tex ® Patch-(V) | 81.46 ± 22.28 | 14%-15% | 179.89 ± 22.00 | 39%-40% |
| CorMatrix ® Patch-(H) | 55.55 ± 26.72 | 14%-15% | 35.53 ± 7.08 | 39%-40% |
| CorMatrix ® Patch-(V) | 39.52 ± 6.78 | 14%-15% | 23.08 ± 12.81 | 39%-40% |
| CardioCel ® Patch-(H) | 13.17 ± 6.59 | 14%-15% | 84.68 ± 29.05 | 39%-40% |
| CardioCel ® Patch-(V) | 10.88 ± 3.76 | 14%-15% | 160.53 ± 25.41 | 39%-40% |

Mechanical characteristics of commercial patches (Gore-Tex®, CorMatrix® and CardioCel®) were obtained under the same conditions as the native tissues and are presented in Table 1. Compared to native tissues, the commercial patches are generally much stiffer, with a tensile modulus in the range of 6-120 MPa at strain=15% in C-direction and 23-180 MPa at strain=40% in R-direction. Commercial patches also display a non-anisotropic behavior, with similar tensile modulus at the same strain level in the horizontal (H) and vertical (V) directions (Table. 1, FIG. 2D-G). And Gore-Tex® is the most isotropic and stiffest samples among these three commercial patches. CorMatrix® and CardioCel® are more compliant than Gore-Tex® and CardioCel® even has a similar tensile modulus as those of HAV at strain=15% in H-direction. They are also relatively anisotropic due to the nature of bio-based patches, with residue fibers in the product. Overall, the commercial patches still possess much stiffer tensile properties compared to HAV or any native leaflets, especially at the R/V direction. Without specific design, they are either randomly anisotropic (for CorMatrix® and CardioCel®) or isotropic (for Gore-Tex®).

Referring to FIG. 2, which shows mechanical properties of native leaflets tissue and the commercial cardiac patch representatives. Tissue specimens were cut from homograft aortic valve and porcine aortic/pulmonary valves (FIG. 2A). The tissue leaflets were cut in circumferential direction and radial (R) direction to prepare specimens for the mechanical test (FIG. 2B). Stress-strain up-curves of HAV, PAV and PPV in C and R directions. Those are average up-curves of 15 cycles after 5 cycles preconditioning. All tissue samples have a much stiffer tensile performance in C direction than R direction (FIG. 2C). None of the three types of commercial patches (Gore-Tex®, CorMatrix®, and CardioCel®) displayed anisotropic mechanical properties (similar average curves in H and V directions) or similar range of tensile modulus close to HAV (FIGS. 2D-2F). And a detailed elastic modulus comparison illustrated the significant difference on mechanical stiffness, between commercial patches and HAV. (FIG. 2G) (*P<0.05, ***P<0.001 and ns=not significant).

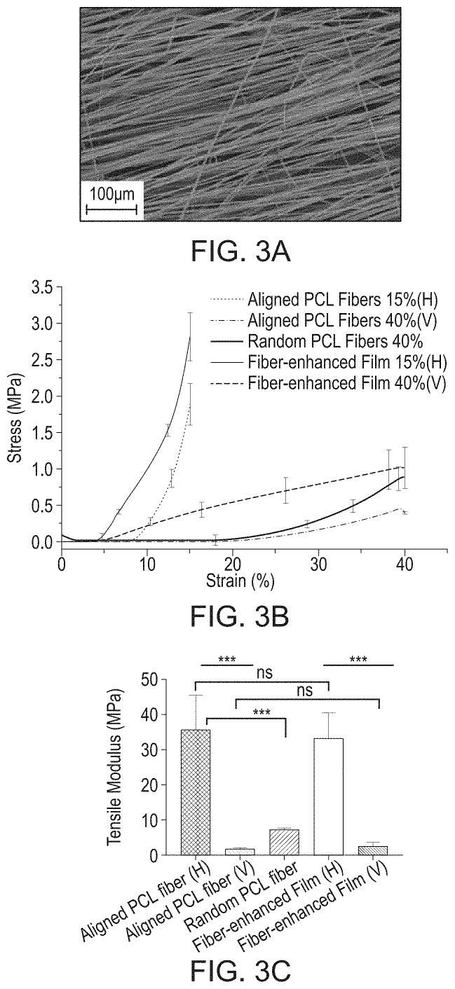

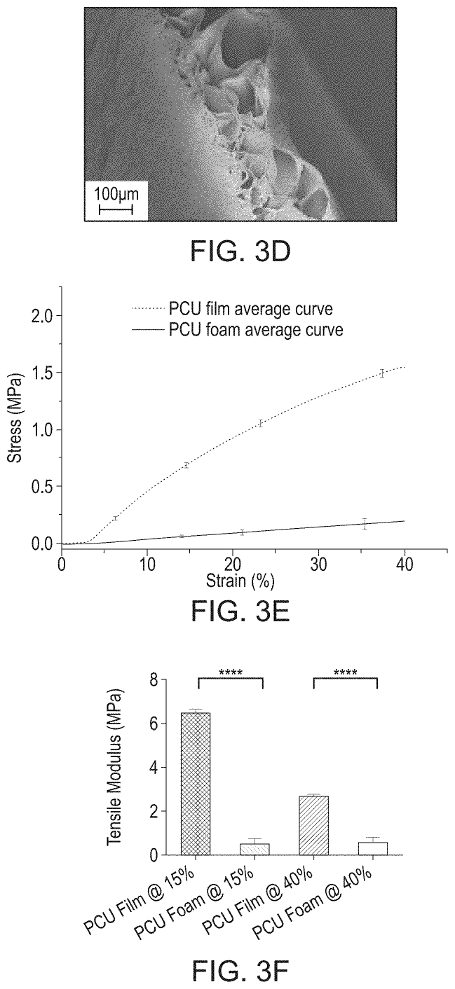

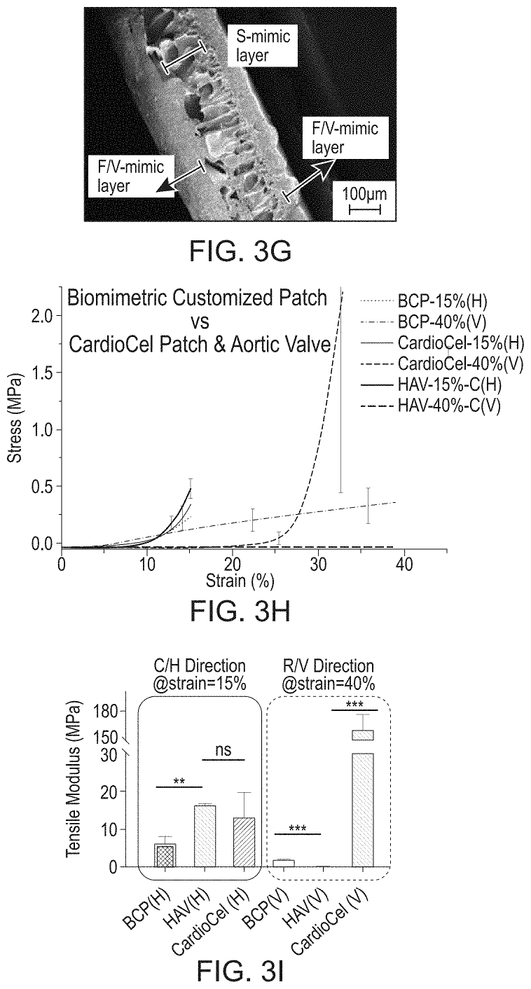

Structure and tensile properties of the mimic layers and the BCPs. FIG. 3 and Table 2 displayed the microscopic structure and mechanical properties of the PCL electrospun fibers, the mimic layers and the BCPs. As demonstrated by the SEM image (FIG. 3A), the aligned PCL fibers are electrospun with a highly-orientated distribution and exhibit a highly anisotropic performance during the cyclic tensile tests (35.74±9.81 MPa vs 1.63±0.38 MPa), compared to the random PCL fibers electrospun from the same solution (7.37±0.30 MPa). The fiber-enhanced F-mimic and V-mimic layers also demonstrate an anisotropic behavior in two directions, due to the incorporation of the aligned PCL fibers (FIG. 3B, 3C). In the H direction, V-mimic and F-mimic layers show a combination of the properties of the PCU film (up-curve) and the PCL aligned fibers (FIG. 3B). While in the vertical direction, they had a similar behavior as the PCU films (FIG. 3E). The PCU properties were dominant and the PCL fibers played a less significant contribution in V direction. The fiber-enhanced layers tensile modulus are 33.38±7.1 MPa at the strain=15% in the H direction and 2.55±1.02 MPa at the strain=40% in the V direction: these layers exhibit an anisotropic behavior and are more compliant than most of commercial patches (except for CardioCel® in H direction) but are still stiffer than the native tissue.

In order to increase the compliant of the overall composite, also to correspond to the Spongiosa layer, PCU foam was made via lyophilization. Compared to the film made from the same concentration PCU solution, the foam exhibited a porous structure (FIG. 3D), a more compliant mechanical behavior and a significant lower tensile modulus (0.55-0.59 MPa), shown in FIGS. 3E and 3F.

The biomimetic, customized three-layered composite patch (BCP) was obtained by coating the fiber-enhanced layers on both sides of the S-mimic layer (FIG. 3G). This BCP demonstrates an anisotropic mechanical behavior which is close to those of native human valve leaflets (FIG. 3H). The BCP also has a tensile modulus of 6.20±1.83 MPa at the strain=15% in the H direction and 1.80±0.21 MPa at the strain=40% in the V direction. Compared to the CardioCel® patch with the best mechanical performance so far, the BCP has a lower stiffness in H direction and more compliant performance in V direction. From the mechanical viewpoint, it is more comparable to the native leaflets than the commercial patches, especially in V/R direction (FIG. 3I, Table 1 and Table 2)

TABLE 2
Tensile Properties of Electrospun Fibers, F/S/V-mimic layers and BCPs

| | | Strain (%) | Tensile | |
|---|---|---|---|---|
| | Tensile Modulus | where to obtain | Modulus | Strain (%) |
| | (MPa) | the | (MPa) | where to obtain the |
| Sample | C/H Direction | tensile modulus | R/V Direction | tensile modulus |
| Aligned PCL fiber | 35.74 ± 9.81 | 14-15% | 1.63 ± 0.38 | 39%-40% |
| Random PCL fiber | 7.37 ± 0.30 | 39%-40% | 7.37 ± 0.30 | 39%-40% |
| Fiber-enhanced Layer | 33.38 ± 7.1 | 14%-15% | 2.55 ± 1.02 | 39%-40% |
| (F/V-mimic layer) | | | | |
| PCU Film | 6.48 ± 0.17 | 14%-15% | 2.70 ± 0.10 | 39%-40% |
| PCU foam | 0.55 ± 0.22 | 14%-15% | 0.59 ± 0.23 | 39%-40% |
| (S-mimic layer) | | | | |
| BCPs | 6.20 ± 1.83 | 14%-15% | 1.80 ± 0.21 | 39%-40% |

Referring to FIG. 3 showing structures and mechanical behaviors of electrospun fibers, fiber-enhanced layers, S-mimic layers and composite patches. The SEM of the aligned PCL fibers have illustrated fibers' orientation (FIG. 3A). Compared to the random PCL fibers, the aligned PCL fibers demonstrated an anisotropic mechanical performance. The fiber-enhanced film (F/V-mimic layers) exhibited an anisotropic mechanical performance, stronger on the H direction (same as aligned fiber direction) and similar performance as pure PCU film on the V direction (perpendicular to the aligned fiber direction) (FIG. 3B-3C). The SEM image displayed the cross-section of the PCU foam: the porous structure and the layer structure (FIG. 3D). The PCU foam has a significant lower elastic modulus (****P<0.0001) compared to the PCU film fabricated from the same solution (FIG. 3E-3F). The SEM image of the cross-section of the composite patch illustrated the tri-layer structure: Film-Foam-Film, which paralleled the design of our composite patch in FIG. 1B (FIG. 3G). The BCP exhibited an anisotropic behavior and a relatively close mechanical performance to HAV in both C/H and R/V directions. Although it may not as stiff as CardioCel in C/H direction, it was more complaint in R/V direction to avoid severe mismatch issue (FIG. 3H-3I).

Flexural properties. Bulge tests were performed to assess the flexural properties of the HAV, commercial patches and the BCP disclosed and embodied herein. The bulge test measured the components of the displacements in a 3D coordinate (FIG. 4C), providing the U, V and W components of the displacement field in X, Y and Z directions. For tissue samples, the X axes were defined as the dominant fiber direction and the Y axes were the perpendicular direction. For commercial patches or BCPs, the X and Y axes were defined by the in-plain directions that corresponded to the stiffest and most compliant performance, respectively. Preconditioning was found to have a negligible effect on the mechanic response. The fiber orientation and anisotropic degree was characterized and evaluated by the principal strains e1 and e2. And the flexural modulus was calculated through the change of the applied pressure (ΔP) and the change of the out-of-plane displacement component (ΔW).

Preconditioning was found to have a negligible effect on the mechanical response. FIG. 4D plots the strain variation at the direction of Z, for the whole 30 loading cycles for the human aortic valve sample. The averaged maximum strain in Z direction over the first 10 cycles was 288.10±1.24%, and the averaged maximum strain over the whole 30 cycles was 289.64±1.40%. It's a 0.53% variation and indicates that preconditioning minimally affected the mechanical response of the tissue. Consequently, the 16^{th} curve was used as the average data to calculate the anisotropic degree and the flexural modulus.

Anisotropic level. FIGS. 4E-G plots the three displacements in X, Y and Z directions at the maximum pressure of the 16^{th} loading cycle and the change of e₁ and e₂ during the 16^{th} loading cycle for the human aortic valve sample as the demonstration representative (FIG. 4H). The ratio between e₁ and e₂ was then defined as the anisotropic level. If the ratio is close to 1, the specimen behaves more like isotropic material. Otherwise, it behaves more like anisotropic material. FIG. 4G exhibited that the contour of the out-of-plane displacement, W, formed concentric ellipses rather than concentric circles. It is also evidence to demonstrate its anisotropy since most of the specimens deformed from a circular sheet to an ellipsoidal dome indicating the presence of anisotropy. Deformation in U and V with different displacement range was also an indication to display the anisotropy of the human aortic valve tissue (FIGS. 4E & F).

Referring to FIG. 4A, a speckled specimen was glued and embedded between the plates (FIG. 4A). Schematic of pressure loading regimen. After 30 s held under a pressure of 0.2 kPa, the samples were loaded from the baseline pressure to a maximum pressure of 7.2 kPa at a rate of 3.5 kPa/s and return to the baseline pressure at the same rate. Total load-unload cycles were 30 (FIG. 4B). Definition of the specimen coordinate system. For tissue samples, X was defined as the dominant fiber direction, Y was defined as the perpendicular direction and Z was defined as the out-of-plain direction. For non-tissue samples, X and Y were defined as the directions with the stiffest and most compliant mechanical performance. Z was also the out-of-plain direction (FIG. 4C). Results of cyclic loading for representative specimen, HAV leaflets at a loading/unloading rate of 3.5 kPa/s. With the similar strain variation in Z direction, the preconditioning had a negligible effect on the long-term mechanic response (FIG. 4D). Contours of displacement components in X, Y, and Z directions at the maximum pressure. The anisotropy of the tissue was evident from the U and V contours and elliptical W contours (FIGS. 4E-4G). Results of the representative cycle of HAV deformation. The change of e1 and e2 were recorded to compare and assessed the anisotropy of the specimen (FIG. 4H).

Table 3 summarized the ratio of principal strain and second principal strain in-plane, e₁/e₂. Among the three commercial patches, the Gore-Tex® patch was the most isotropic one, and CorMatrix® is the most anisotropic. For native tissues, PPV and HAV have obvious anisotropic behaviors. For BCPs, although from the design and the tensile test data they were demonstrated as the anisotropic composites, the average ratio of e₁/e₂ is just higher than Gore-Tex®. It may be attributed to the similar scale of the tensile modulus in-plane X and Y directions.

TABLE 3
Flexural Properties of Native Tissues, Commercial Patches and BCPs

| | Thickness | | | | |
|---|---|---|---|---|---|
| Sample | (mm) | ΔW (mm) | ν | e₁/e₂ | E_{flex} (MPa) |
| Gore-Tex ® | 0.382 ± 0.011 | 0.120 ± 0.031 | 0.46 | 1.16 ± 0.13 | 17.58 ± 4.50 |
| CorMatrix ® | 0.309 ± 0.109 | 0.366 ± 0.030 | 0.45 | 1.80 ± 0.68 | 10.52 ± 1.03 |
| CardioCel ® | 0.364 ± 0.101 | 0.722 ± 0.137 | 0.45 | 1.52 ± 0.13 | 4.52 ± 2.40 |
| PPV | 0.271 ± 0.032 | 1.275 ± 0.266 | 0.45 | 2.10 ± 0.81 | 4.87 ± 1.59 |
| PAV | 0.480 ± 0.001 | 1.922 ± 0.077 | 0.45 | 1.29 ± 0.04 | 0.53 ± 0.02 |
| HAV | 0.347 ± 0.038 | 1.196 ± 0.472 | 0.45 | 1.70 ± 0.51 | 2.70 ± 1.30 |
| BCP | 0.688 ± 0.186 | 0.231 ± 0.166 | 0.33 | 1.26 ± 0.02 | 3.55 ± 2.80 |

All data is acquired from the 16^{th} loading-unloading cycles during the bulge tests

Flexural Modulus: Table 3 also summarized the data of thickness and displacement of specimens in the out-of-plane direction. It can be seen that the commercial patches generally possessed higher flexural modulus: Gore-Tex® was the stiffest among those three types of patches (17.58±4.50 MPa) and CardioCel® was the most compliant one (4.52±2.40 MPa). Native tissues, including porcine leaflets and human leaflets, behaved more compliant than commercial patches during the bulge tests. For BCPs, they had a similar flexural modulus range (3.55±2.80 MPa) as HAV (2.70±1.30 MPa), and displayed better compliance than Gore-Tex® and CorMatrix®.

Suture retention: The resistance to tearing of the BCPs, the raw material (PCU film) and the three commercial patches were determined by suture retention strength measurements. The mean suture retention strength (SRS) of Gore-Tex®, CardioCel® and CorMatrix® are 5.35±1.25 N, 8.99±1.77 N and 4.07±1.38 N respectively (Table 4). The SRS of the BCP and the PCU film are in the range of the commercial patches (FIGS. 5C-5D). Moreover, there is no significant difference on SRS of composite patches in H and V directions, reflecting a uniform resistance to tearing on the whole patch.

A thickness-normalized SRS (TN-SRS) has also been applied to eliminate the effect of sample thickness and needle size. According to the Equation 5, the TN-SRS of Gore-Tex®, CardioCel® and CorMatrix® were 94.76±22.14 N/mm², 98.26±19.35 N/mm², and 82.86±28.10 N/mm² respectively (Table 4). There's no significant difference on TN-SRSs among those three commercial patches. TN-SRSs of the BCPs in two directions were 89.91±13.25 N/mm², and 79.1±11.1 N/mm² respectively and were not significantly different from the three commercial patches (FIG. 5F). It was noted as well that the BCPs had a longer elongation, around 20 mm (FIG. 5E) than the commercial patches, which indicated that the composite patch had a higher toughness than other samples by simply integrating the stress-strain curves.

TABLE 4
Suture Retention Strength and Thickness-Normalized Suture
Retention Strength for All Samples

| Sample | Thickness (μm) | SRS (N) | TN-SRS (N/mm²) |
|---|---|---|---|
| PCU Film | 242.5 ± 1.5 | 6.32 ± 1.02 | 175.53 ± 28.33 |
| Gore-Tex ® Patch | 383 ± 4.9 | 5.35 ± 1.25 | 94.76 ± 22.14 |
| CardioCel ® Patch | 603.3 ± 27.3 | 8.99 ± 1.77 | 98.26 ± 19.35 |
| CorMatrix ® Patch | 344 ± 11.4 | 4.07 ± 1.38 | 82.86 ± 28.10 |
| BCP (H) | 509 ± 21.5 | 6.58 ± 0.97 | 89.91 ± 13.25 |
| BCP (V) | 509 ± 21.5 | 6.25 ± 0.88 | 79.1 ± 11.1 |

Referring to FIG. 5 shows Suture Retention Strength and Thickness-normalized Suture Retention Strength. Example (FIG. 5A) and schematic representation (FIG. 5B) of a specimen during suture retention strength test: The main geometrical parameters and the tensile rate are defined. Representative SRS curves of the commercial patches, the PCU films and the BCPs (FIG. 5C). Comparison of all groups displayed that the BCP had a SRS in the range of the average level of the commercial patches. Although One-way ANOVA test displayed the difference (*p<0.05) between BCP and commercial patches, the following Tukey's test verified no significant difference between BCP and each commercial patch. The difference was found among the commercial patches (FIG. 5D). Representative TN-SRS curves of the commercial patches, the PCU films and the BCPs (FIG. 5E). The TN-SRS of the BCP had no significant difference compared to the commercial patches (FIG. 5F).

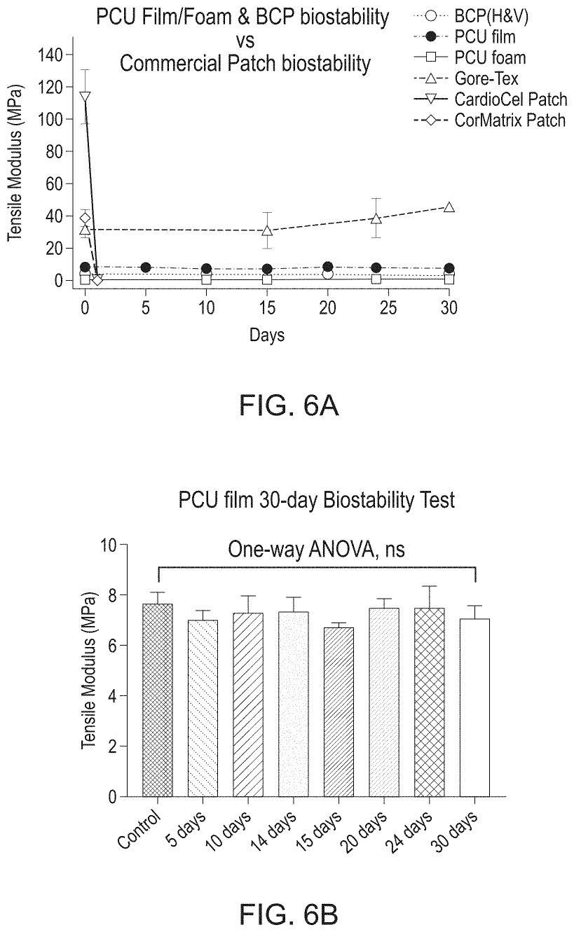

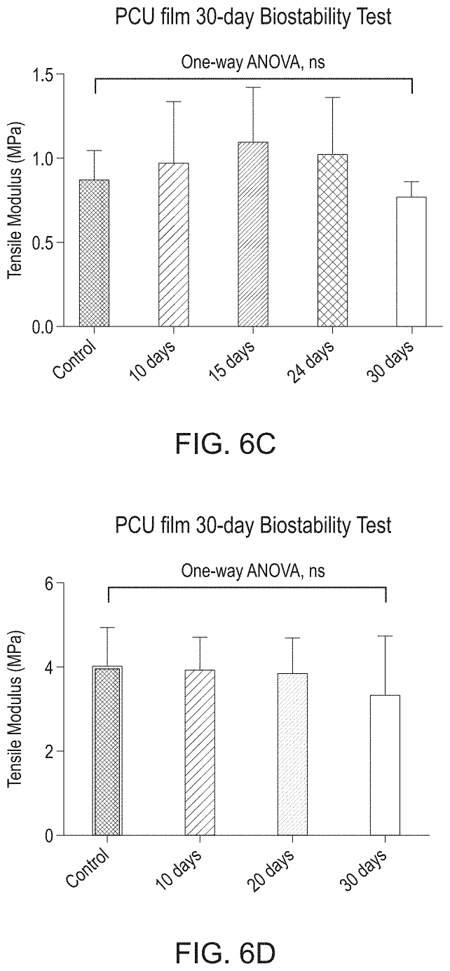

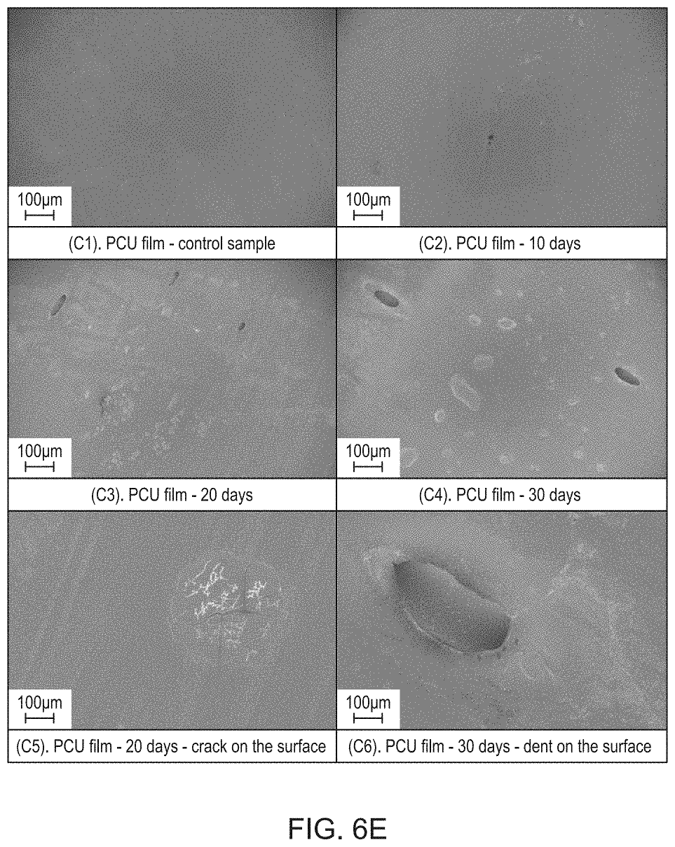

Biostability. The biostability of the commercial patches, PCU-based raw film/foam and our BCPs were assessed by an accelerated oxidative degradation test, using a 0.1 M CoCl₂/20% H₂O₂ solution. FIGS. 6A-E show the results of the biostability tests applied to all samples. Two of the commercial patches, CorMatrix® and CardioCel®, which are derived from biological materials, fully degraded and dissolved in the oxidization solution within Day 1, while the polymer-based samples (Gore-Tex®, PCU film/foam and BCPs) display an excellent stability during the 30-day period (FIGS. 6A-D), no significant difference on the mechanical properties in 30 days (one-way ANOVA). Nevertheless, the SEM images of PCU films' surface display the formation of oxidization spots and dents after 20-30 days (FIG. 6C), suggesting a potential that the BCPs, with PCU film as the outside layer, might be impacted by a long-term oxidization starting from the surface.

Referring again to FIGS. 6A-E, biostability performance of three commercial patches, the PCU films/foams and BCPs is shown. The tensile modulus of polymer-based product, including PCU film/foam, Gore-Tex® and BCP remained stable throughout the 30 days in the accelerated oxidization solution, as opposed to the commercial CardioCel® and CorMatrix® patches, demonstrating an excellent biostability for a duration equivalent to 15 months of in vivo implantation (FIG. 6A) Specifically, the PCU films and foams as the main component and BCP itself, showed a stable mechanical performance (no significance change on mechanical properties via One-way ANOVA) in 30 days (FIG. 6B-6D SEM images unveiled details on the surface morphology, suggesting the beginning of a slow degradation process starting at the outside surface layer (FIG. 6E).

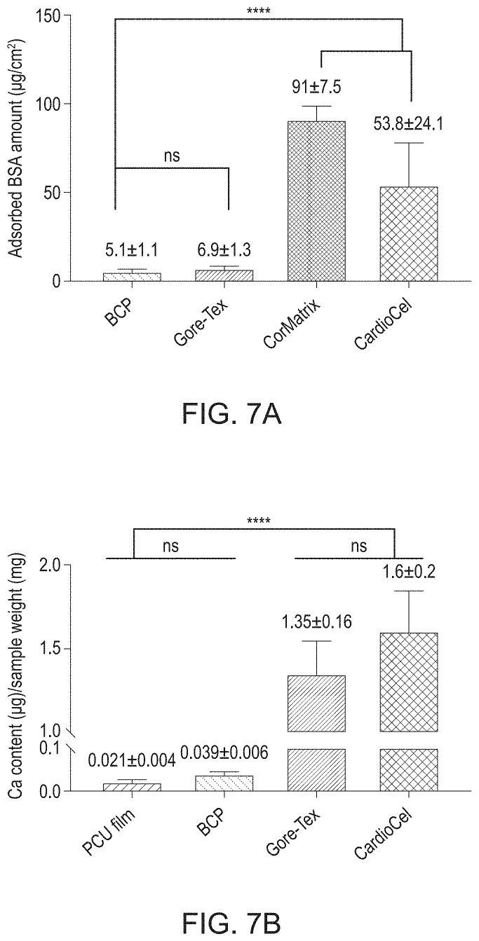

Biocompatibility. BSA Protein Adsorption. A BSA protein adsorption test was applied to assess the blood compatibility of the three commercial patches and BCPs. FIG. 7A illustrates the amounts of adsorbed protein on the BCPs and three commercial patch surface. The two polymer-based patches (BCP and Gore-Tex®) showed similarly low adsorbed BSA amounts and no significant difference between the adsorption levels of these two samples. On the other hand, the two patches derived from biological tissues (CorMatrix® and CardioCel®), exhibit a much higher dose of adsorbed albumin compared to the BCPs (p<0.0001, One-way ANOVA). The BCPs, thus, has a low level of protein adsorption that compares favorably to the three commercial patches.

Ca²⁺ Adhesion. Table. 5 shows the results of 16-day Ca²⁺ adhesion tests performed on the PCU film, BCP and commercial patches. It has clearly stated that the PCU film and BCP have a lower Ca²⁺ deposition compared to Gore-Tex® and CardioCel® patches (FIG. 7B). A similar trend has been observed in the calcification amounts from the 8-week in vivo rat subcutaneous test (Table 5). Overall, both of the BCP and PCU surface layer have a lower level of calcification compared to commercial patches in vitro tests. It also provides a solid foundation for further evaluation in vivo tests.

Referring to FIG. 7 showing biocompatibility performance: BSA protein adsorption and Ca²⁺ adhesion of commercial patches, the PCU film and the BCP. The polymer-based sample, BCP and Gore-Tex® patch had a significant lower amount of BSA protein adsorption compared to two biological materials-derived patches (****p<0.0001). No significant difference on the capacity of BSA absorption between BCP and Gore-Tex® (FIG. 7A) In a 16-day in vitro Ca-ion adhesion test, the PCU film and BCP had a lower Ca contents in unit of dry samples, compared to the Gore-Tex® patch and the CardioCel Patch®, which demonstrated that BCP and its main component had a better resistance to calcification than commercial patches (****p<0.0001) (FIG. 7B).

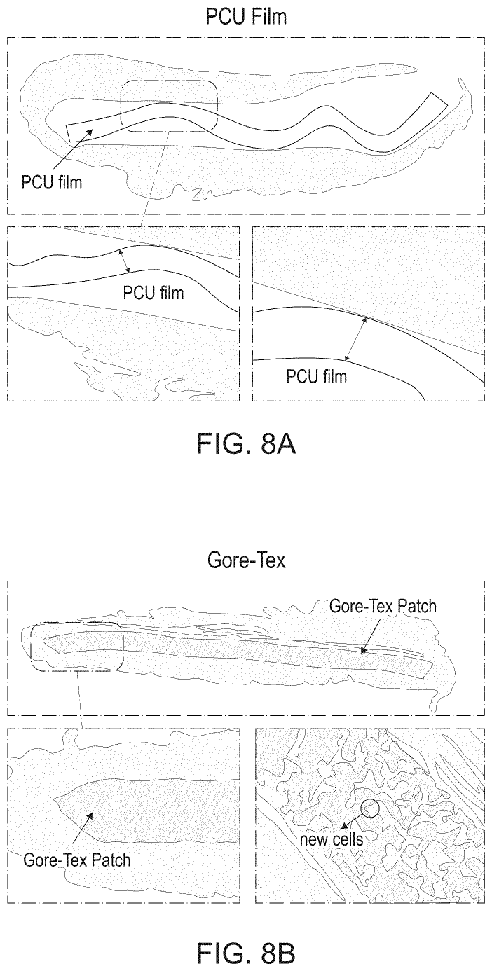

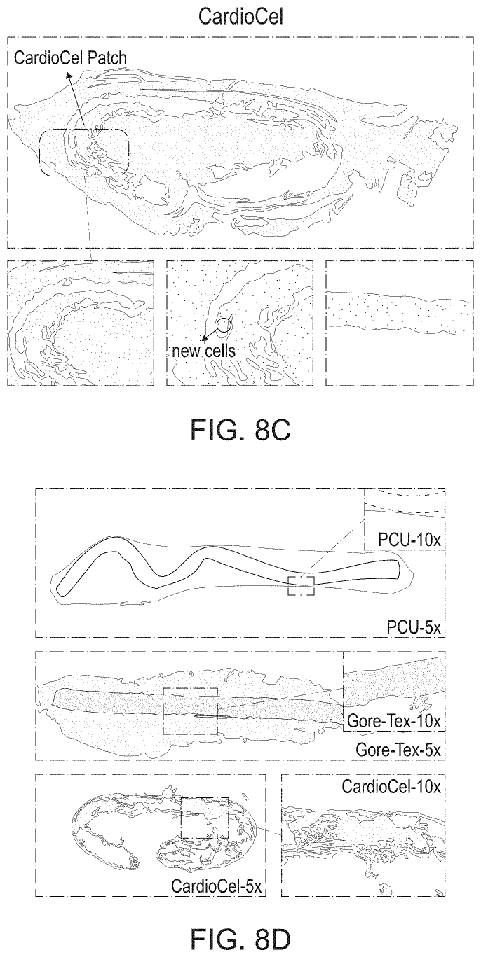

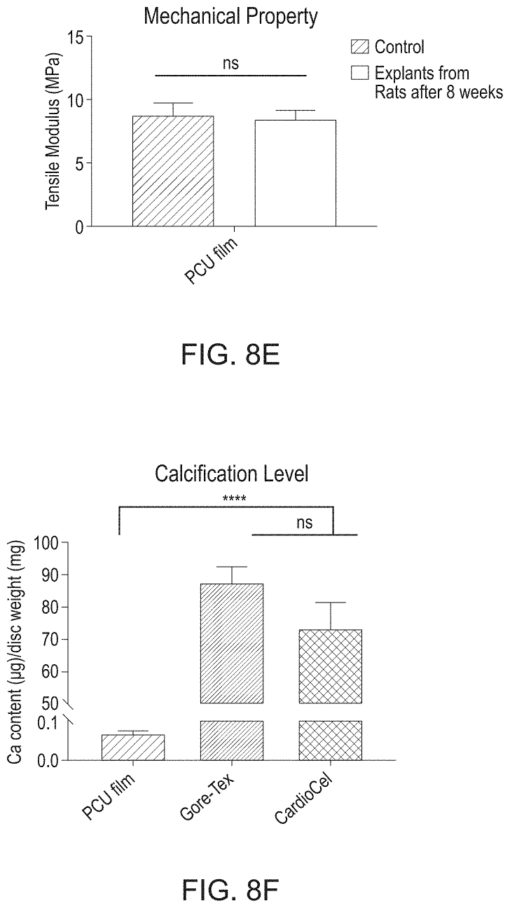

In vivo studies. Subcutaneous implantation. A set of schematic illustrations of H&E images from three samples: PCU film, Gore-Tex® and CardioCel® Patch, are presented in FIG. 8A-8D. Two polymeric samples, the PCU film and the Gore-Tex® patch, had a layer of tissue capsuled tightly at the interface and both of them kept a relatively intact morphology (FIG. 8A (PCU film) VS FIG. 8B (Gore-Tex® patch)). For the PCU film, the specimen had delaminated with the adjacent neo-tissue during microtome cutting due to the elastic properties in comparison with the surrounding tissue. There was no cell or tissue growth into the PCU film, whereas cell infiltration occurred in the Gore-Tex® patch. CardioCel® Patch, on the other hand, displayed a different tissue response: first, the patch had a severe degradation. It was hard to observe the intact CardioCel® compared to the control sample (FIG. 8C). Second, cellular nuclei were found in the residue CardioCel® Patch (FIG. 8C, bottom, middle) and those were from the adjacent tissues, which indicated the cell infiltration and tissue growth into the patch.

PCU film had no evidence of calcification as indicated by FIG. 8D (top). A high degree of red staining appeared in two commercial patches, indicating calcification in the explant patches and the interface between the encapsulated tissue and the patch sample (red staining is shown as shading in schematic FIGS. 8D (middle) and 8D (bottom)). Little to no calcification was present in most part of the encapsulated tissue.

Subsequently a calcium content assay was conducted and confirmed the histological findings. A significant increase in Ca²⁺ level was found in Gore-Tex® and CardioCel® samples compared to the PCU film, with p<0.0001 (Table 5. and FIG. 8F). Moreover, there was no significant difference in tensile modulus of the PCU film before and after implantation (FIG. 8E).

TABLE 5
Calcium Colorimetric Assay Results for the PCU Film, BCP and
Commercial Patches

| | Calcification amount | Calcification amount |
|---|---|---|
| | 16-day In vitro Ca²⁺ Adhesion | 8-week In vivo Rat |
| | test | Subcutaneous Test |
| Sample | (μg/mg) | (μg/mg) |
| PCU Film | 0.022 ±0.005 | 0.067 ± 0.006 |
| BCP | 0.039 ±0.008 | — |
| Gore-Tex ® Patch | 1.35 ± 0.19 | 87.7 ± 4.7 |
| CardioCel ® Patch | 1.60 ±0.24 | 73.26 ± 8.13 |

Referring back to FIG. 8A-8D, histological characterization, mechanical property and calcium quantification of the PCU film, Gore-Tex® patch and CardioCel® Patch after in vivo implantation is shown. Sections of the PCU film, Gore-Tex® and CardioCel® had a layer of tissue capsuled at the interface. No cell infiltration or tissue growth within the PCU film but cellular nuclei were found in Gore-Tex® and CardioCel®. CardioCel® had a sign of degradation and cannot obtain an intact morphology (FIGS. 8A-C).

Sections of three samples also displayed the distribution of calcification (red color shown as shading in the schematic illustrations) in tissues and the patch samples. No visible calcification appeared in PCU specimen but a high degree of calcification presented in two commercial specimens (131-133) FIG. 8D. There was no significant change on mechanical property before and after in vivo implantation (ns, via t-test) (FIG. 8E). Calcium quantification data clearly demonstrated PCU film has a significantly better resistance to calcification in vivo subcutaneous model, with *p<0.0001, compared to the Gore-Tex® patch and CardioCel® Patch (FIG. 8F).

Utilizing the biostable and biocompatible polymers as the main components, described herein is a polymer-based, tri-layered patch to mimic the three-layer architecture of native leaflets. The in vitro and in vivo assessment of our BCPs covers two main parts: the long-term mechanical and biological performance.

The mechanical assessment utilizes the cyclic uni-axial tensile tests, flexural bulge tests and suture retention tests for characterization. Tensile test offers a more direct and more economical approach to characterize the mechanical properties. Studies on the uniaxial tensile properties of valve leaflets in the literature have stretched the specimens to break, and recorded the ultimate stress (MPa), the strain-to-failure/ultimate strain (%), as well as calculated the elastic modulus (MPa) using the Equation 1. The ultimate stress and stain-to-failure were acquired beyond the physiological level and unveiled the properties which were not fit the working range; and the one-time tensile stretch cannot reflect the performance at steady state, especially considering the fact that the initial tensile curve behaves more differently from the rest of cyclic curves of viscoelastic materials due to the Mullins' effect and the preconditioning effects. Tensile modulus, was commonly used to easily quantify an intrinsic elastic property of soft, viscoelastic biomaterials. Note that the stress and strain in the tensile modulus were engineering stress and engineering strain, so the effect of the cross-sectional contraction was not reflected in the tensile modulus.

A 20-time cyclic tensile test for all the samples was conducted. The maximum strain was set as 15% in H/C direction and 40% in V/R direction, corresponding to the physiological level from systole to diastole. The averaged, post-conditioning curves was picked to do the calculation of tensile modulus to eliminate the influence of the Mullin's effect and the preconditioning effects. In order to compare BCP with reference tissues and commercial patches, the tensile modulus was calculated at the strain of 15% and 40% for the H/C-direction and V/R-direction, respectively.

The averaged tensile curves and modulus data display that HAV is stiffer than PAV, and PPV is stiffer than PAV. It was also found that the anisotropic behavior and matched mechanical properties at the specific strain range were hardly achieved in commercial patches. Most of them are either too stiff (except for CardioCel® in H direction) or isotropic, compared to the HAV. They are far from the satisfactory material to match the native tissue, from the mechanical view.

BCP, thus, was designed and fabricated using solution casting, lyophilization and electrospinning to replicate the complex, structure-function driven architecture of native leaflets. It was demonstrated that a patch with such structure (FIG. 1B) was able to mimic the anisotropic mechanical properties of the native tissue. The aligned PCL fibers were embedded in the PCU film to mimic the fibrosa and the ventricularis. Indeed, the anisotropic properties of the native leaflets come from the orientated dense collagen bundles and elastin network that exist in these two layers. The spongiosa, however, is inherently soft and compliant with a much lower stiffness. Thus, a foam structure made of PCU was designed to mimic the spongiosa. Utilizing the lyophilization, the ice in the frozen-PCU film was removed under the low pressure and the framework inside was kept to maintain its porous structure. This porous structure was demonstrated to confer flexibility and the shock-absorbing properties, as well as offered a relatively lower mechanical stiffness to tailor the BCP. Combining the two fiber-enhanced layers and foam together, the BCP exhibited a tensile modulus of 6.20±1.83 MPa at the strain=15% in the H direction and 1.80±0.21 MPa at the strain=40% in the V direction. Compared to commercial patches, this BCP, for the first time in the literature, to the best of our knowledge, demonstrated the mimic architectures, anisotropic behaviors and tensile modulus (elasticity) much closer to the human valve leaflets.

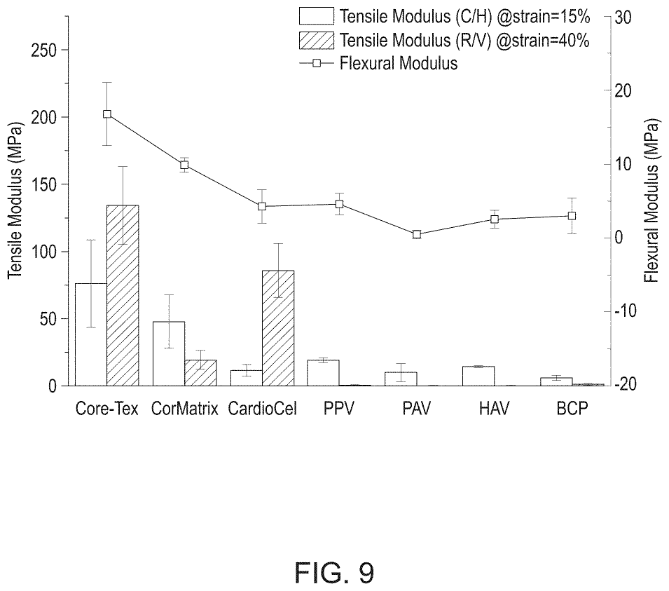

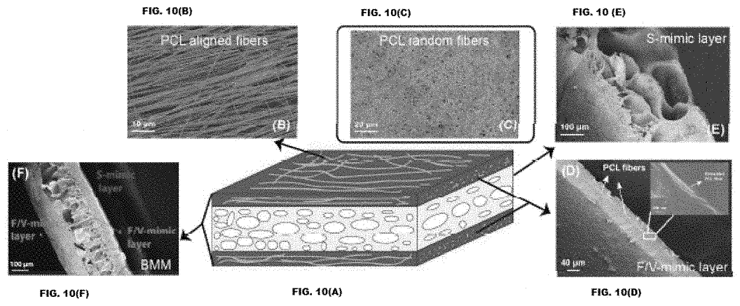

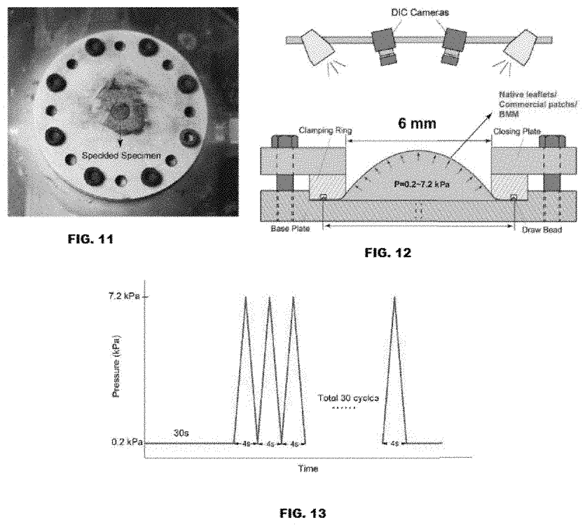

The flexural properties of the BCP, heart valve tissues and commercial patches, were also studied using the bulge tests. Due to the limitation of the pressure transducer and the capacity of the customized syringe pump, the maximum pressure can reach 7.2 kPa (54 mmHg) as a valid, stable level and a frequency of 0.25 Hz allows for specimen inflation. The results from each specimen still demonstrated the various performance on flexural deformation under a quasi-physiological simulation. Three commercial patches displayed a randomly anisotropy performance and higher flexural modulus (4.52-17.58 MPa). Gore-Tex® is made of ePTFE and has no particular design for anisotropic applications. It leads to an isotropic behavior during the tests. While CorMatrix® and CardioCel® are derived from bio tissues and it is reasonable to have some residual fibers in the patch, which provide anisotropy. For the BCP, due to the similar scale of the tensile modulus in-plane X and Y directions, it didn't display an obvious anisotropic performance in-plane. It also emphasizes the significance to decrease the modulus of the BCPs in V/R direction in order to compare with native tissue level. All of HAV, PAV and BCP have a lower flexural modulus between 0.53-3.55 MPa. This performance is also in line with the trend of tensile modulus data shown in Table 1 and 2, especially the one in C/H direction as shown in FIG. 9. Gore-Tex® and CorMatrix® are much stiffer than native tissues and BCPs. CardioCel®, although much stiffer at strain=40% in V direction, has a compliant performance in H direction and can be compared with HAV and BCP in tensile test and bulge test. From the flexural property view, BCPs offer a good option to be used as alternative patches with a flexural modulus matching the native leaflets. It also demonstrates the bulge test is valid to acquire flexural properties for further systole-diastole hydrodynamic study and simulation.

Suture retention capability. Punctures and defects are generated during suturing, which may result in mechanical failure through crack propagation. Therefore, the resistance to tear, characterized as SRS and TN-SRS, are essential to evaluate the feasibility of the patches or alternatives. From the results it can be seen the SRS of our BCP and its raw materials (6.25-6.58 N) were in the range of the ones of commercial patches (4.07-8.99 N), which demonstrates that they have a similar capacity of resistance to tearing as the commercial products. It is noted that a number of different suture thread thicknesses and needle types were applied in the clinics, depending on the detailed applications and surgeons' selection. Some geometrical parameters such as the diameter of the suture, the thickness of the graft wall remain unconstrained by the norm. Thus, TN-SRS was also introduced to evaluate the suture retention capability of the products, normalizing this parameter without impact from the product and thread thicknesses. The TN-SRS of BCP has no significant difference from the ones of commercial patches. And BCP also has a higher toughness than most of commercial patches, which emphasizes its durable nature. To sum up, a series of suture retention tests demonstrated that the BCP has a resistance to tearing similar, even better than the commercial patches, no matter from the SRS, TN-SRS or toughness.

The biological assessment of the BCPs and commercial patches includes the biostability and biocompatibility, in vitro and in vivo. As a designed, polymer-based patch, it is expected to be stable in vivo and the mechanical properties do not alter over time. Published papers reported that the degradation of polyurethane-based materials in vitro and in vivo was attributed to several mechanisms including metal ion-induced accelerated oxidative degradation, hydrolytic degradation and enzymatic degradation. It is demonstrated that oxidative degradation was the more dominant mechanism over other degradations. Thus, a 0.1 M CoCl₂/20% H₂O₂ solution was applied in this test to accelerate oxidative degradation of the PCUs. The Co²⁺ ions have been demonstrated to rapidly decompose hydrogen peroxide via the Haber-Weiss reaction. Degradation results after 24 days in this solution was shown to correlate to 12 months of in vivo implantation. The modulus of the BCP and PCU film/foam displayed no significant change (NS, One-way ANOVA) on mechanical properties in 30 days in this accelerated oxidization solution. It demonstrated that the BCP has a stable performance which was equivalent to 15 months of in vivo implantation. Even so, a slow oxidative degradation sign was found on the outside surface layer. This finding suggests that the biostability of the BCP, although being comparable to the one of FDA-approved Gore-Tex® patches, may be improved down the road through a surface modification process targeting the resistance to oxidation.

To evaluate biocompatibility, protein adsorption and calcium-ion adhesion are selected to assess BCP and commercial patches' biological performance in vivo. Protein adsorption is a significant factor to determine the thrombogenicity of an implanted graft. When blood gets in contact with the graft's surface, protein adsorption occurs first, then leads to more plugs aggregation, eventually provokes the generation of the fibrin network and thrombus formation. Thus, our BCP should aim at reducing their potential for protein adsorption and cut the path of forming thrombin. Bovine serum albumin has a structure similar to human serum albumin (HSA) and the HSA has the highest concentration in human plasma. A BSA protein adsorption test was performed to characterize the blood compatibility of the surfaces of our BCPs and the commercial patches. And the BCP exhibited a low level of protein adsorption compared to three commercial patches. It may be attributed to its smooth PCU film surface without holes or sites, which avoids the plugs deposition and formation.

On the other hand, it is significant to evaluate the resistance to calcification when developing any biomaterial since calcification is the leading reason of failure of bioprosthetic heart valves and grafts. It is a complex phenomenon influenced by a series of mechanical and biochemical factors. It also limits the durability of synthetic polymer materials used in heart valve devices and blood contact application in general. In vitro Ca²⁺ adhesion tests using a MCP solution to mimics the hydroxyapatite level were performed. A 16-day test exhibits that the BCP and its main component PCU film have a lower level of Ca²⁺ ion accumulation compared to commercial patches. And this trend is also in line with the findings from in vivo subcutaneous tests (FIG. 7D). The BCPs displays a slightly higher mean value than the film, which may be attributed to its porous S-mimic layer embedded between films offering more sites on the side for Ca²⁺ ions accumulation. And it is reasonable to infer the BCP should have a slightly higher calcification level than pristine PCU film but much lower than commercial patches.

An in vivo rat subcutaneous implantation has been conducted to verify the biostability and biocompatibility of PCU film (the main composition of BCPs) and two commercial patches. The former exhibited a stable performance and little/no cell or tissue infuse or grow within the patch. It also exhibited a little-to-no calcification level, better than commercial patches. No obvious mechanical properties degradation after the tests and the tissue generated around the PCU patch were organized and no-calcification. It is a good sign to highlight the feasibility to apply the PCU-based BCP in vivo and expect the positive outcomes.

Compared to three commercial patches, this BCP demonstrated an anisotropic mechanical behavior and mechanical stiffness (6.20±1.83 MPa and 1.80±0.21 MPa in circumferential and radial directions, respectively), which was much closer to the native aortic valve leaflets than any currently available commercial patches. What's more, our BCPs also showed an excellent durability in an in vitro accelerated oxidization solution and displayed an excellent biocompatibility with an in vitro lower protein adsorption level and a lower calcium adhesion level. In vivo rat subcutaneous tests confirmed its main composition, PCU's mechanical biostability and superior resistance to inflammation and calcification, compared to the commercial patches.

The native-like performance of the BCP avoids patch failure and degeneration, which are related to the inadequate mechanical properties. It is biostable, and does not rely on uncontrolled polymer degradation and tissue formation. The biomimetic patch also exhibits a low protein adsorption and low Ca²⁺ adhesion, avoiding a high risk of thrombogenicity and calcification. In some embodiments, fiber meshes can be fabricated by various biocompatible polymers, to optimize the anisotropic mechanical performance.

In some embodiments, the biostability and biocompatibility is optimized through adding the surface layer on the current version, for example, Parylene C can be evenly coated on the patch through chemical vapor deposition.

The biomimetic patch is scalable. For example, at a lab scale, this version of the patch is processed through solution casting, electrospinning and lyophilization. A multiple technology combination provides flexible tuning methods for optimization. At an industrial scale, this tri-layer composite can be fabricated via a non-expensive and scalable multi-layer co-extrusion technology. This green, non-solvent involved method provides a better reproducibility and lower costs of production. It provides a feasible path to commercialize this polymeric patch to improve the durability and quality of the valve repair, and decrease the number of reoperations and complications. In some embodiments, the surface morphology is further processed to create the “corrugations” structure to mimic the native leaflet's surface morphology. This structure plays an important role and accounts for the native collagen fiber's mechanical behavior during valve closing.

Example: A polycarbonate urethane-based material with aligned polycaprolactone fibers to enhance the anisotropic properties are disclosed. Solution casting, electrospinning and lyophilization were used to mimic the native leaflet's architecture. Compared to current commercial materials, this BMM exhibited an anisotropic behavior and a mechanical performance much closer to the native aortic leaflets. The material exhibited biostability in an accelerated oxidization solution equivalent to 15 months of implantation. It also displayed better resistance to protein adsorption and calcification in vitro and in vivo. This material is shown to have long-term durability for surgical valve repair or replacement.

Materials: Carbothane™ AC-4075A, Polycarbonated-based polyurethane (PCU) (Lubrizol, Wilmington, Mass.) was dissolved in dimethylacetamide (DMAC) (Acros Organics, Fair Lawn, N.J.). Polycaprolactone (PCL, Mw=80,000; Sigma-Aldrich, St. Louis, Mo.) was used to create fibers and dissolved with a mix of chloroform (Sigma-Aldrich, St. Louis, Mo.) and methanol (Fisher Scientific, Hampton, N.H.) with a 3:1 molar ratio. Three commercially available patches were selected for comparison: Gore-Tex® (W. L. Gore and Associates, Flagstaff, Ariz., USA), CorMatrix® (Cardiovascular, Inc, Atlanta, Ga., USA) and CardioCel® (Admedus, Toowong, Queensland, Australia). The CryoValve® aortic human valve (CryoLife Inc., Kennesaw, Ga., USA) was used as the control sample, after being dissected and kept intact in PBS.

Fabrication of the biomimetic multilayered material (BMM). Fibrosa-mimic layer and Ventricularis-mimic layer fabrication: Using a 15% PCL solution prepared in a mixed solvent, PCL fibers were produced by electrospinning with the following parameters: a flow rate of 1 ml/hour, a voltage of 20 kV voltage and a distance of 15 cm between the nozzle and drum collector. The solution was spun towards a rotating collector at a rate of 1600 rpm to collect the aligned fibers. The fibers were dried overnight in a chemical hood 192 for solvent evaporation. The collected, aligned PCL fibers were embedded in a solution-casted PCU film. The 15% PCU solution was casted by a doctor-blade coater through a 500 μm gap to control the film thickness. The fiber-solution composites, fibrosa-mimic (F-mimic) layer and ventricularis-mimic (V-mimic) layer, were cured overnight in a chemical hood to evaporate the solvent and form the fiber-enhanced layers.

Spongiosa-mimic layer fabrication: 15% PCU solution was casted by a doctor blade coater to create a film with a fixed thickness of 1500 μm. Subsequently, the film was immersed in deionized water for 24 hours in order to replace the solvent with water. The film was frozen at −80 and lyophilized at 0.1 mBar and −40 for 72 hours, leading to the formation of a porous structure (or foam)

BMM fabrication: The F-mimic layer was used as the bottom layer of the BMM. It was fabricated first as described above. After 1 hour drying in the hood, the spongiosa-mimic (S-mimic) layer was placed over the half-cured composite and fully cured with this F-mimic layer overnight. Then this two-layer composite was stacked on top of the V-mimic layer to fabricate the three-layered BMMs using the same strategy.

Morphology characterization. The specimens (PCL aligned fibers, each mimic layer and the BMMs) were sputter coated with gold/platinum and imaged with a Zeiss Sigma VP scanning electron microscope (SEM) at an accelerating voltage of 3 kV. SEM images were used to assess the fibers' orientation, mimic layers and the BMMs' structures and surface morphology.

Tensile mechanical tests. Mechanical tests were performed using an Instron 5848 mechanical tester with a 50 N load cell at a strain rate of 10% s-1. The specimens were cut as 5 mm×20 mm stripes (for non-tissue samples) or 3 mm×10 mm ones (for the native tissue samples) in two different directions, circumferentially (C-direction) and radially (R direction). The thickness was measured at three different points with a digital caliper (Mitutoyo America Corp, Aurora, Ill., USA) and the values were averaged. Four to six specimens for each sample were repeatedly stretched for 20 cycles, either to a maximal strain of 15% in the C-direction or to a maximal strain of 40% in the R-direction. Missirlis and Chong, Brewer et al, Thubrikar et al. and Li et al. have all reported in vivo AV leaflet strains of physiological level to be approximately 10-15% and 30-40% in the circumferential and radial directions respectively. After the first 5 preconditioning cycles, the subsequent 15 cycles of stress-strain curves were recorded and averaged and the tensile modulus E were calculated as the Equation 1, discussed herein.

Flexural mechanical tests. Samples for the flexural mechanical tests were cut as planar specimens with enough area to fully cover the test hole (diameter=6 mm). Thickness was evaluated by averaging three measurements taken at specimen's center with a digital caliper. The specimens were speckled with black India ink to allow for digital image correction (DIC) tracking deformation and glued between two plates with holes of 6 mm diameter (FIG. 11). The embedded specimen was secured onto a custom inflation chamber through the holder (FIG. 12).

Specimens were inflated by a custom-made displacement-driven syringe injection of PBS into the custom-made pressurization chamber. The pressure was monitored by a pressure transducer with a range of 0-8 kPa. The loading regimen was programmed using Lab View (V2020, National Instruments, Austin, Tex.). The specimen was brought to a baseline pressure of 0.2 kPa and held for 30 seconds prior to cyclic testing to ensure the 250 specimen was at equilibrium. The specimens were subjected to 30 load-unload cycles at a rate of 3.5 kPa/s from the baseline pressure to a maximum pressure of 7.2 kPa (FIG. 13) to mimic the leaflet deformation during the cardiac cycle. The deforming specimen surface was imaged by two stereoscopically arranged cameras with 20 mm focus lengths at an aperture of f/4. The optical axes of the cameras were positioned 35 cm above the chamber and fixed with a total angle of 12°. This configuration had a depth of field in front over 1.5 cm, sufficient to capture the deformation. Images were collected during testing at a rate of 10 Hz by VicSnap 2009 and correlated by Vic3D (V8, Correlated Solution, Inc. Columbia, S.C., USA).

The flexural bulge test measured the components of the displacements in a 3D coordinate plane, providing the U, V and W components of the displacement field in X, Y and Z directions. The elastic modulus measured with this flexural bulge test, E_{flex}, was calculated through the change of the applied pressure (ΔP) and the change of the out-of-plane displacement component (ΔW). The sample in this test was modeled as a circular thin plate with edges fully fixed. The pressure was evenly distributed on the bottom surface of the sample. The governing equation and boundary conditions of this case could be expressed in cylindrical coordinates (r, as in Equation [2] described herein. The solution to this equation is derived as equation [3] described herein. At the center point (=0), Eflex. is expressed as equation [4]. Where ΔW is the change of the displacement in z direction. Here, all the materials were assumed to be incompressible, so the Poisson's ratios were all set as 0.5.

Suture retention tests. The suture retention tests were conducted using an Instron 5848 tensile machine. Prolene 5-0 suture was inserted 2 mm from the end of the 10×15 mm specimen and through the specimen to form a half loop. The suture was pulled at a rate of 50 mm/min crosshead speed. Five specimens were tested in each group. The force (N) required to pull the suture through and/or cause the specimen to fail was recorded as the suture retention strength (SRS). A thickness normalized suture retention strength (TN-SRS, N/mm2) was also applied to eliminate the effect of sample thickness and needle size. TN-SRS is calculated by dividing the suture retention strength by the area of the sample over which the load was applied, Equation [5] as described herein.

Biostability tests. Specimens were pre-cut as 5 mm×30 mm and submerged into 2 mL vials filled with an in vitro solution of 20% hydrogen peroxide (H2O2)/0.1M cobalt chloride (CoCl₂). The in vitro solution was refreshed twice a week, and all tests were done at 37° C. After a period of 5, 10, 14, 15, 20, 24, and 30 days, the specimens were removed, rinsed thoroughly in deionized water, dried in the hood, then cut into two parts (5 mm×25 mm and 5 mm×5 mm). The former was tested via the tensile tests and the modulus at strain=15% was calculated. The latter was analyzed by SEM to inspect the surface quality.

Biocompatibility tests. Bovine Serum Albumin (BSA) static protein-adsorption experiments. For static protein-adsorption tests, 1 mg mL-1 BSA solution was prepared in PBS (pH 7.4). BMMs and commercial patches were cut into specimens (50 mm×10 mm) and immersed in 10 mL 1 mg mL-1 BSA solution in a test tube. BSA adsorption was conducted under vibration at 37° C. for 3 hours to allow for adsorption equilibrium. Then the specimens were rinsed with PBS, and the remaining proteins adsorbed on the surfaces were removed with a 1 wt % aqueous solution of sodium dodecylsulfate (SDS), as described by Song et al. The experiments were performed with five measurements for each specimen. BSA content was measured using a NanoDrop™ spectrophotometer at a wavelength of 280 nm, and then the amount of adsorbed BSA on specimens was calculated.

Calcium deposition experiments. The calcium deposition experiments were performed in a metastable calcium phosphate (MCP) solution. The MCP solution has been previously described in detail. In brief, 3.87 millimole (mM) CaCl₂), 2.32 mM K₂HPO₄ and 0.05M Tris buffer were solved in 1000 ml of de-ionized water, to yield a Ca/PO₄ ratio of 1.67. This solution is more physiologically representative of hydroxyapatite, which is the most common form of calcium minerals in the vascular calcification process. BMM, PCU film and commercial patches were cut into specimens (5 mm×30 mm) and immersed in 2 mL MCP solution individually. This experiment was conducted under vibration at 37° C., and the solution was changed every 48 hours to ensure an adequate ion concentration. The specimens were removed after 16 days and rinsed with water to remove excess solution and loosely attached deposits. The specimens were dried in the vacuum oven at 70° C. overnight, weighed and hydrolyzed in 2 mL of 2 N HCl for 24 hours at 50° C. The calcium concentration was determined from HCl hydrolysate, using a calcium colorimetric assay.

Rat subcutaneous implant model. In accordance with NIH guidelines for the care and use of laboratory animals (NIH Publication #85-23 Rev. 1985), all animal protocols were approved by the Institutional Animal Care and Use Committee (IACUC) of Columbia University (Protocol #AC-AABD5614).

Eighteen specimens (diameter 333=8 mm) of PCU film (n=6), Gore-Tex® patch (n=6) and CardioCel® patch (n=6) were implanted in the subcutaneous position of three rats. Following induction of anesthesia, fur clipping, standard sterile prepping and draping, six subcutaneous pockets were created on the dorsal surface of each rat. One specimen was implanted into each pocket, after which all wounds were re-approximated with surgical clips. The rats were sacrificed at 8 weeks with an overdose of isoflurane (Euthenase).

Histology. The implanted specimen was retrieved while still contained in host tissue, fixed in 10% neutral buffered formalin and processed using paraffin-embedding techniques. Slides were stained with Hematoxylin and Eosin and Alizarin Red stains. In each specimen, both the patch and the surrounding host tissue were evaluated.

Calcium content & mechanical test. Samples were analyzed for calcium content using calcium colorimetric assay as described above regarding biocompatibility tests. Briefly, the specimen disks were removed from host tissue, fixed in formalin and solvent-exchanged in DI-water. Following the lyophilization, the net weight of the specimen disks was acquired. After hydrolyzing in nitric acid, the calcium content was quantified (microgram calcium per milligram dry specimen weight). The PCU disks were separated from the host tissue after lyophilization. This specimen's mechanical performance was also evaluated as described above regarding tensile mechanical testing, and its tensile modulus at the strain=15% was recorded, to compare with the control, unimplanted sample.

Statistical Analysis. For studies including various groups of samples, like the tensile property studies, the suture retention tests, the biocompatibility studies (protein adsorption and calcium deposition), etc., two-sided t-tests for parametric data with Welch's correction were conducted and used for analysis. The biostability studies, which included the same group of BMM samples, were analyzed using the one-way ANOVA followed by Tukey's post-hoc tests (GraphPad Prism 7, San Diego, Calif., USA). Results are showed as means±standard deviation. P values less than 0.05 were considered statistically significant (*P<0.05, **P<0.01, ***P<0.001 and ****P<0.0001).

The results of the testing are discussed herein. Structure. The BMM was designed as a tri-layer polymeric structure that was specifically developed to mimic the tri-layer anatomy of the native valve (FIG. 10(A)): an F-mimic layer, an S-mimic layer and a V-mimic layer. FIG. 10(B) shows that the aligned PCL fibers predominantly exist with a highly-orientated distribution, while random PCL fibers are electrospun with a random direction (FIG. 10(C)). The cross-section image (FIG. 10(D)) shows the aligned fibers were embedded in the PCU film to form two fiber-enhanced layers (F-mimic and V-mimic layers). PCU foam is used as the S-mimic layer showing a porous structure created after lyophilization in the cross-sectional view (FIG. 10(E)). The three mimic layers were tightly bound together via “glue”—PCU solution used for F-mimic layer and V-mimic layer—to form the BMM, with two fiber-enhanced layers outside and the foam layer inside (FIG. 10(F)).

Tensile properties. Cyclic, uniaxial tensile tests were performed to assess the tensile properties of BMM and its component layers (Table 6).

TABLE S
Tensile Properties of BMM and its Mimic Layers, Native Tissue,
and Commercial Patches

| | Tensile Modulus (MPa) | | Tensile Modulus (MPa) | |
|---|---|---|---|---|
| Sample | strain = 15% | | strain = 40% | |
| BMM | 6.20 ± 1.83 (C) | | 1.80 ± 0.21 (R) | |
| Aligned PCL | 35.74 ± 9.81 (C) | | 1.63 ± 0.38 (R) | |
| fiber | | | | |
| Random PCL | N/A | | 7.37 ± 0.30 | |
| fiber | | | | |
| PCU film | 6.48 ± 0.17 | | 2.55 ± 1.02 | |
| Fiber- | 33.38 ± 7.1 (C) | | 2.56 ± 1.02 (R) | |
| enhanced | | | | |
| Layer | | | | |
| (F/V-mimic | | | | |
| layer) | | | | |
| PCU foam | 0.55 ± 0.22 | | 0.59 ± 0.23 | |
| (S-mimic | | | | |
| layer) | | | | |
| HAV | 16.34 ± 0.42 (C) | | 0.03 ± 0.01 (R) | |
| Gore-Tex ® | 72.38 ± | 69.33 ± | 131.17 ± | 135.83 ± 5.26 |
| | 36.68(C) | 20.31 (R) | 33.67 (C) | (R) |
| CorMatrix ® | 47.87 ± | 30.91 ± | 24.81 ± | 36.25 ± |
| | 24.23(C) | 5.42(R) | 0.90(C) | 5.13(R) |
| CardioCel ® | 13.84 ± | 5.23 ± | 66.03 ± | 85.83 ± |
| | 2.74(C) | 2.72(R) | 5.00(C) | 24.50(R) |

For samples that may show certain mechanical anisotropy, they will be tested in two directions, noted as C for circumferential direction and R for radial direction

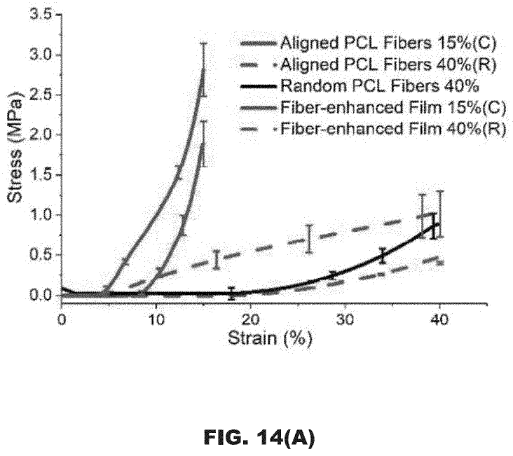

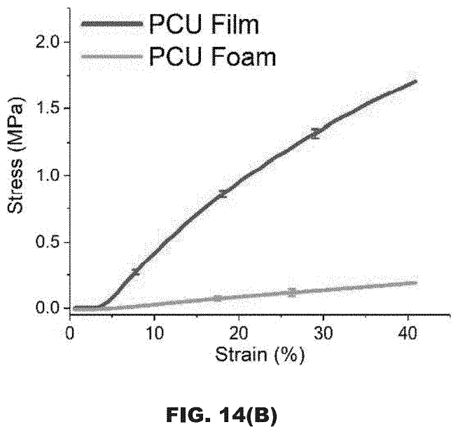

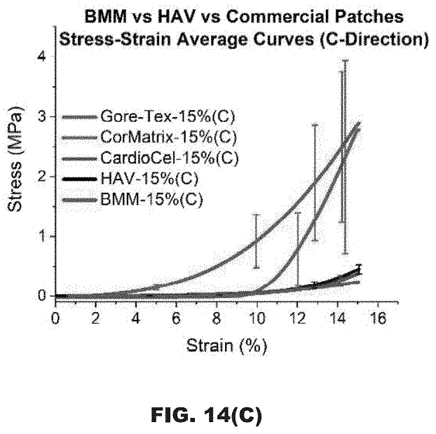

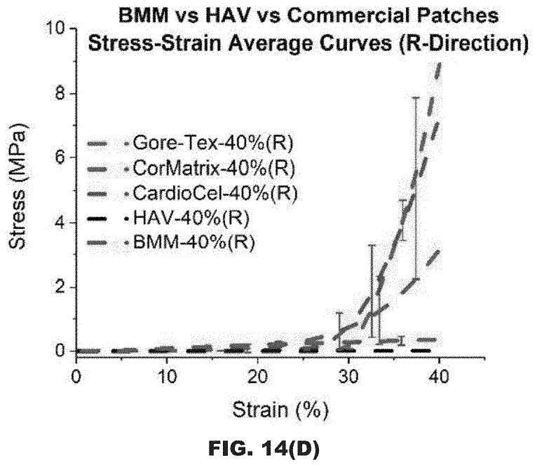

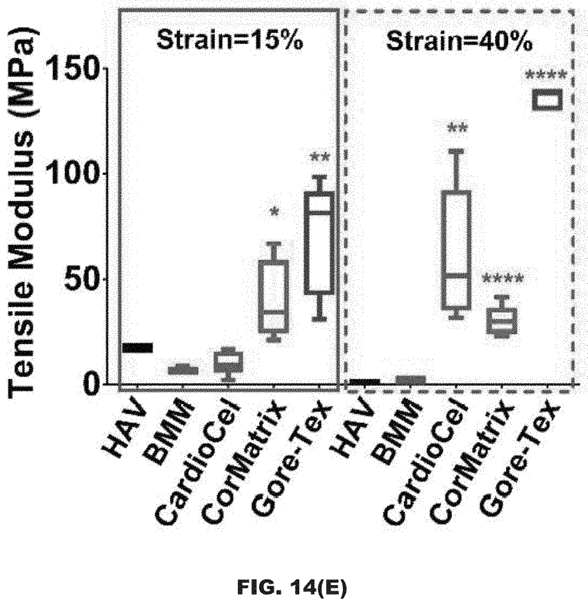

The aligned PCL fibers exhibit a highly anisotropic performance (35.74±9.81 MPa vs. 1.63±0.38 MPa), compared to the random PCL fibers electrospun from the same solution (7.37±0.30 MPa). Due to the incorporation of the aligned PCL fibers, the fiber-enhanced layers also demonstrate an anisotropic behavior, stronger along the fiber-aligned direction (green solid curve) and similar performance to pure PCU film along the fiber perpendicular direction (green dash curve), shown in FIG. 14(A). Conversely, the PCU foam, which is used to mimic the spongiosa layer of the native leaflet, exhibits a more compliant mechanical behavior and a significant lower tensile modulus (0.55±0.22 MPa), compared to the PCU film (6.48±0.17 MPa) made from the same solution (FIG. 14(B)). Combining three layers together, the BMM also demonstrates an anisotropic mechanical behavior and has a tensile modulus of 6.20±1.83 MPa at 15% strain in the C-direction and 1.80±0.21 MPa at 40% strain in the R-direction (FIG. 14(E), Table 6).

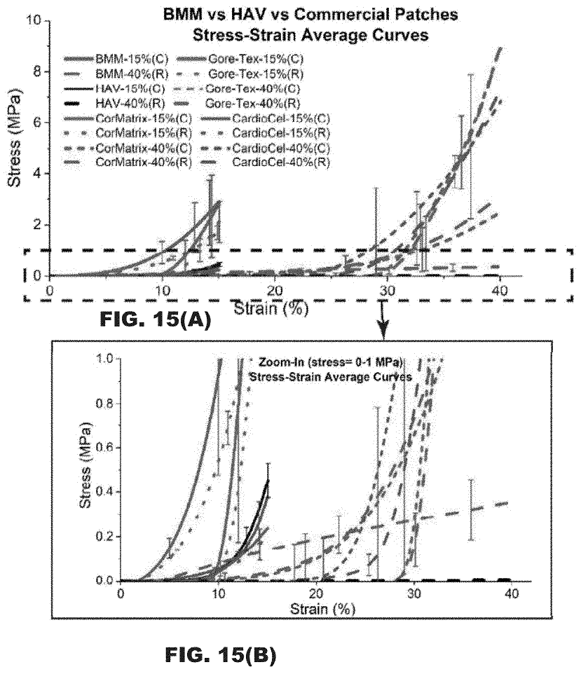

Native tissues and commercial patches were also tested under the same conditions to compare with BMM. For native leaflets, the average stress-strain loading curves exhibit a residue deformation 398 and then an increase in the slope of the stress-strain curves which is attributed to the deformation and stretch of fiber networks. This increase is accentuated in the C-direction compared to the R-direction because of the existence of oriented collagen fibers (FIGS. 14(c) and 12(D), black curves). The human aortic valve leaflets (HAVs) have a tensile modulus value of 16.34±0.42 MPa in the C-direction, while a quite low modulus of 0.03±0.01 MPa in the R-direction (Table. 6 and FIG. 14(E)). On the other hand, three selected commercial patches are generally much stiffer, with a tensile modulus in the range of 6-120 MPa and 23-180 MPa in two orthogonal directions (Table. 6). They also display a non-anisotropic behavior, with similar tensile curves at the same strain level in these two directions (FIG. 15(A)-(B)) Gore-Tex® is the most isotropic and the stiffest sample among the three commercial patches. CorMatrix® and CardioCel® are less stiff, and CardioCel® even has a similar tensile modulus to those of HAV in the C-direction. The latter two samples are also relatively anisotropic given their biological nature and the presence of residual extracellular matrix fibers. Nevertheless, the commercial patches still possess much stiffer properties than HAVs, especially in the R-direction.

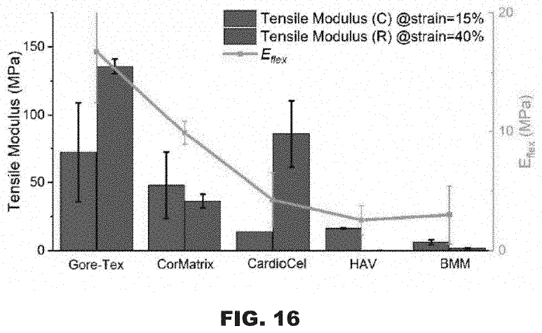

Flexural properties. Bulge tests were performed to assess the flexural properties of the HAV, commercial patches and our BMM. Table 7 summarized the data of thickness and displacement of specimens in the out-of-plane direction. The commercial patches generally possessed higher Eflex: Gore-Tex® was the stiffest among the three commercial patches (16.73±4.28 MPa) and CardioCel® was the most compliant (4.25±2.26 MPa). HAV was more compliant than commercial patches during the bulge tests. BMMs had a similar Eflex range (2.99±2.43 MPa) as HAV (2.54±1.22 MPa), and displayed better compliance than commercial patches. Commercial products have a generally stiffer performance than native tissues and BMMs, whether from tensile tests in two directions or from flexural bulge test (FIG. 16).

TABLE 7
Flexural Properties of BMM, Native Tissue and Commercial Patches

| Sample | Thickness(mm) | W (mm) | Eflex (MPa) |
|---|---|---|---|
| BMM | 0.688 ± 0.186 | 0.231 ± 0.166 | 2.99 ± 2.43 |
| HAV | 0.347 ± 0.038 | 1.196 ± 0.472 | 2.54 ± 1.22 |
| Gore-Tex ® | 0.382 ± 0.011 | 0.120 ± 0.031 | 16.73 ± 4.28 |
| CorMatrix ® | 0.309 ± 0.109 | 0.366 ± 0.030 | 9.89 ± 0.98 |
| CardioCel ® | 0.364 ± 0.101 | 0.722 ± 0.137 | 4.25 ± 2.26 |

All data is acquired from the 16th loading-unloading cycles during the bulge test

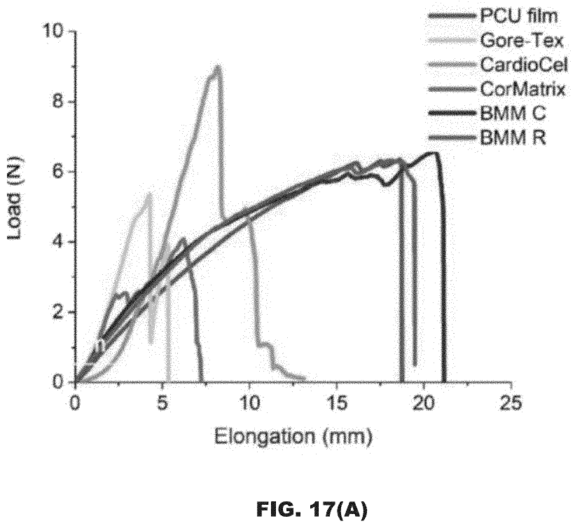

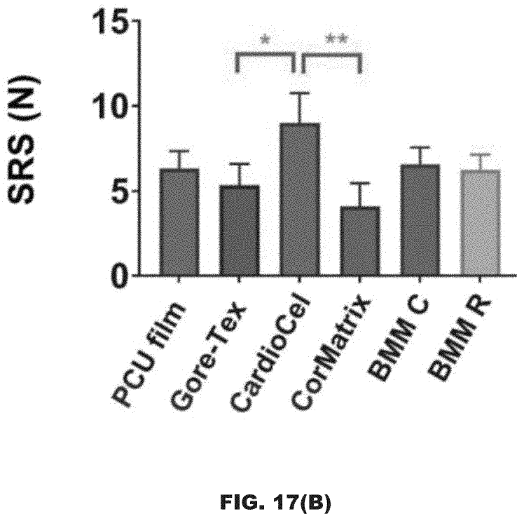

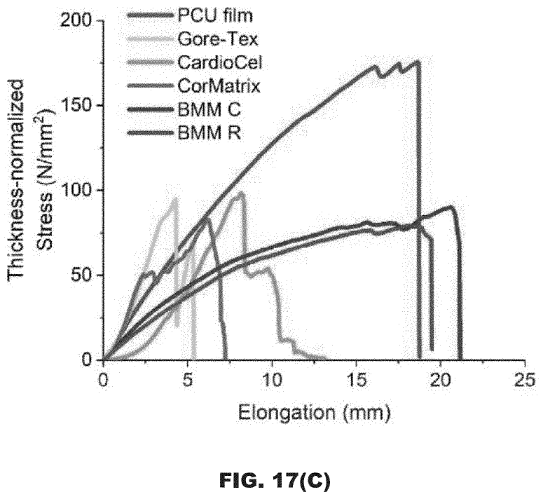

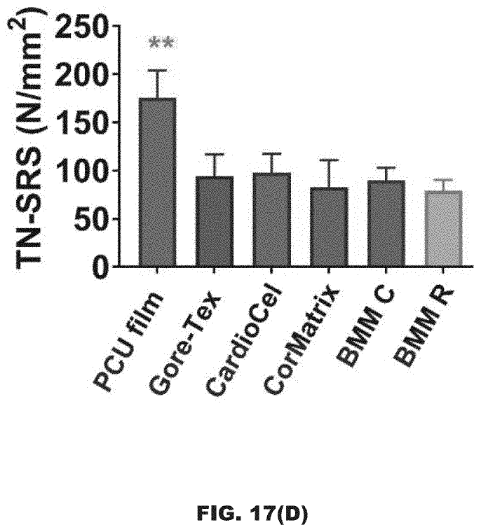

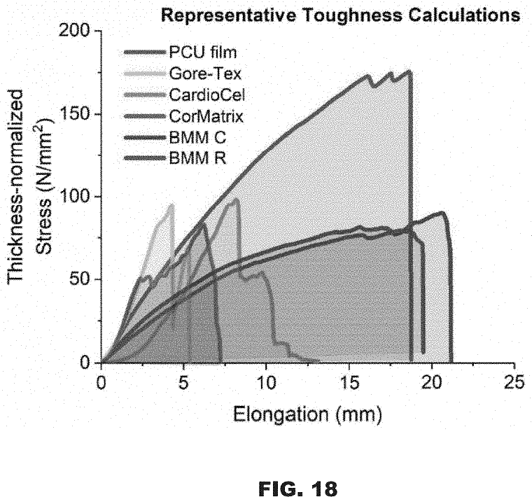

Suture retention of samples. The resistance to tearing of the BMM and its main component, PCU film, compared to the three commercial patches was determined by suture retention strength (SRS) measurements. The mean SRS of the BMM and the PCU film were 6.58±0.97 N and 6.25±0.88 N respectively. There was no significant difference on SRS of the BMMs in two directions, reflecting a uniform resistance to tearing. The mean SRS of Gore-Tex®, CardioCel® and CorMatrix® were 5.35±1.25 N, 8.99±1.77 N and 4.07±1.38 N respectively (FIGS. 17(A)-17(B)). On the other hand, the TN-SRSs of the BMMs in C− and R− directions were 89.91±13.25 N/mm², and 79.1±11.1 N/mm² respectively and were not significantly different from the three commercial patches (FIGS. 17(C)-17(D). By calculating the area under the stress-strain curve, it was also noted that the BMM had a significantly higher toughness than the commercial patches (FIG. 18, Table 8), which indicated that the BMM was able to withstand more energy from tear to fracture.

TABLE 8
Suture Retention Strength, Thickness-normalized Suture
Retention Strength and toughness of BMM, its raw film and
commercial patches

| | | | TN-SRS | Toughness |
|---|---|---|---|---|
| Sample | Thickness (μm) | SRS (N) | (N/mm²) | (J/mm³) |
| BMM(C) | 509 ± 21.5 | 6.58 ± 0.97 | 89.91 ± 13.25 | 1.26 ± 0.20 |
| BMM(R) | | 6.25 ± 0.88 | 79.1 ± 11.1 | 0.99 ± 0.14 |
| PCU Film | 242.5 ± 1.5 | 6.32 ± 1.02 | 175.53 ± 28.33 | 2.04 ± 0.33 |
| Gore-Tex ® | 383 ± 4.9 | 5.35 ± 1.25 | 94.76 ± 22.14 | 0.25 ± 0.06 |
| CorMatix | 603.3 ± 27.3 | 8.99 ± 1.77 | 98.26 ± 19.35 | 0.43 ± 0.08 |
| CardioCel ® | 344 ± 11.4 | 4.07 ± 1.38 | 82.86 ± 28.10 | 0.34 ± 0.12 |

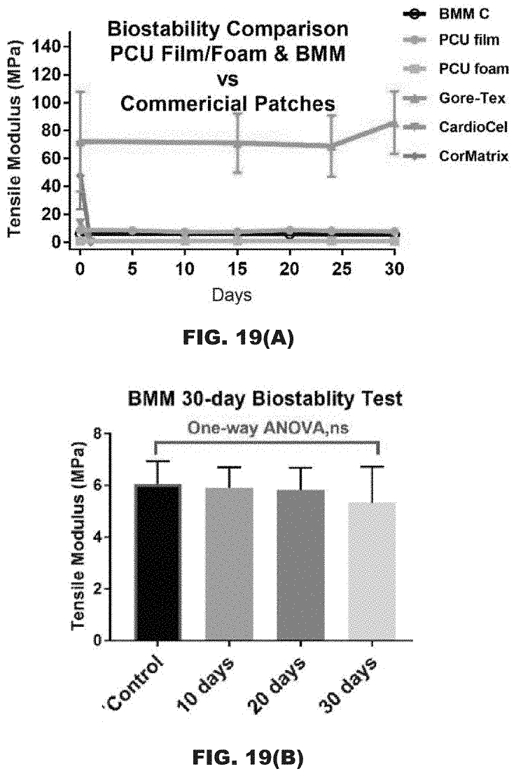

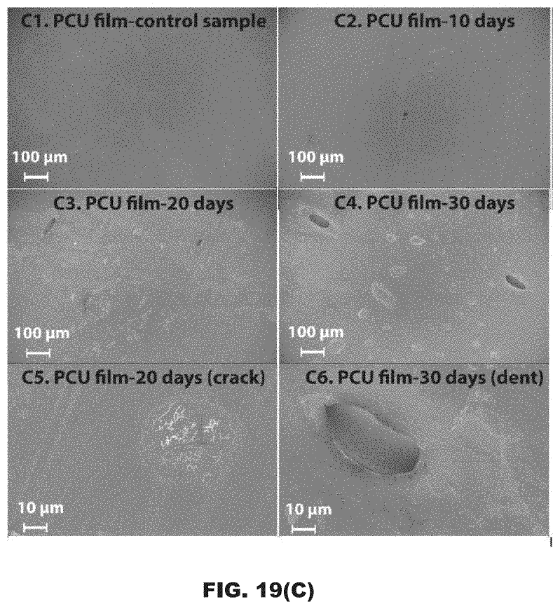

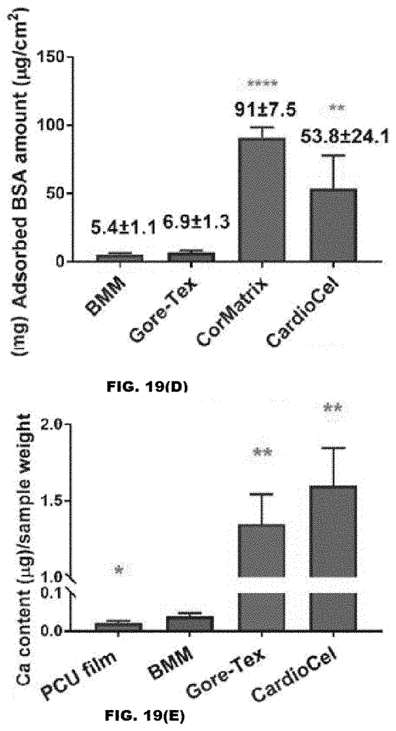

Biostability. The biostability of the BMMs, PCU film/foam, and three commercial patches were assessed via measuring the degradability of samples in the accelerated oxidative solution. The polymer-based samples (BMM, PCU film/foam and Gore-Tex®) remained stable throughout the 30 days in the accelerated oxidization solution (FIG. 19(A)-19(B)) with no significant difference in the mechanical properties. On the other hand, the two tissue-based materials fully degraded in the oxidization solution within Day 1. After 20-30 days, the formation of oxidization spots and dents on the PCU films' surface was apparent, as shown in FIG. 19(C), suggesting the beginning of a slow degradation process starting at the outside surface layer.

Biocompatibility. Bovine Serum Albumin (BSA) adsorption. A BSA protein adsorption test was applied to assess the blood compatibility of the artificial material surface. FIG. 19(D) illustrates the amounts of adsorbed protein on the surface of BMM and three commercial patches. The polymer-based materials (BMM and Gore-Tex®) showed similarly low adsorbed BSA amounts. Conversely, the two tissue-derived patches exhibited a much higher concentration of adsorbed albumin compared to the BMMs. The BMM therefore demonstrates a low level of protein adsorption that compares favorably to the three commercial patches.

Ca2+ deposition To study the material's susceptibility to calcification, the in vitro deposition of Ca2+ ions on BMM, Gore-Tex®, and CardioCel® samples was evaluated in a MCP solution, as previously described. FIG. 19(E) shows that both BMM and its main component material PCU have a lower level of calcification compared to commercial patches in 16-day in vitro tests (Table 9), which demonstrate a better resistance to calcification. It also provides a solid foundation for further evaluation in in vivo tests, shown in the in vivo studies, described herein.

TABLE 9
Calcium Colorimetric Assay Results of the main layers of BMM
and two commercial patches

| | Calcification amount | Calcification amount |
|---|---|---|
| | 16-day In vitro Ca²⁺ | 8-week In vivo Rat |
| | Deposition tests | Subcutaneous Test |
| Sample | (μg/mg) | (μg/mg) |
| PCU Film | 0.022 ± 0.005 | 0.067 ± 0.006 |
| PCU Foam | 0.039 ± 0.008 | — |
| Gore-Tex Patch | 1.35 ± 0.19 | 87.7 ± 4.7 |
| CardioCel Patch | 1.60 ± 0.24 | 73.26 ± 8.13 |

FIGS. 19(A)-19(E) illustrate the biostability and biocompatibility performance of three commercial patches, the PCU films/foams and BMMs. FIG. 19(A) illustrates the tensile modulus change of all samples in 30 days in the accelerated oxidization solution. FIG. 19(B) illustrates the tensile modulus of BMM in 30 days showed a stable mechanical performance (ns=no significance, via One-way ANOVA). FIG. 19(C) illustrates SEM images that show details on the surface morphology of BMM from 0-30 days. FIG. 19(D) illustrates the BSA protein adsorption of the BMM and commercial patches. CorMatrix® and CardioCel® patches that have a higher BSA adsorption level compared to the BMM (****p<0.0001 and **p<0.01). FIG. 19(E) illustrates the Ca deposition of the PCU film, BMM and commercial patches. The PCU film and BMM had a lower Ca contents (PCU vs BMM, *p<0.05) in unit of dry samples, compared to the Gore-Tex® patch (vs BMM, **p<0.01) and the CardioCel® patch (vs BMM, **p<0.01).

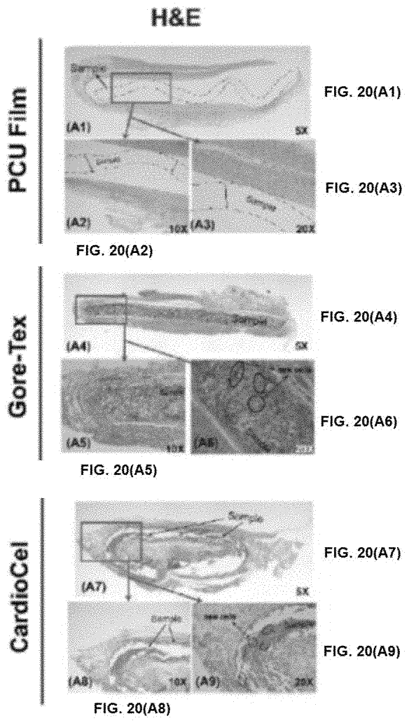

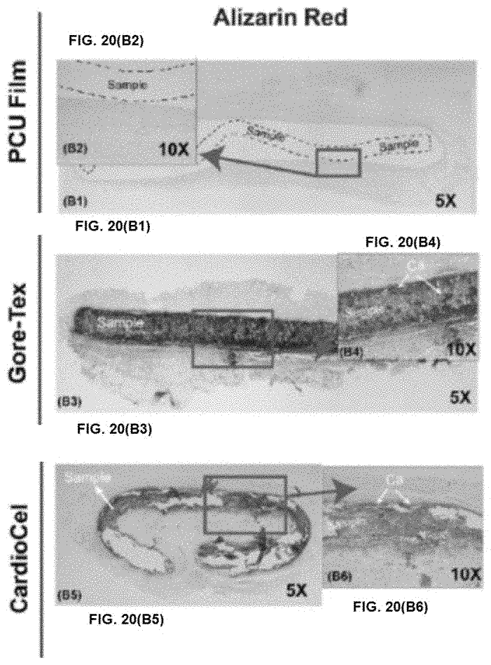

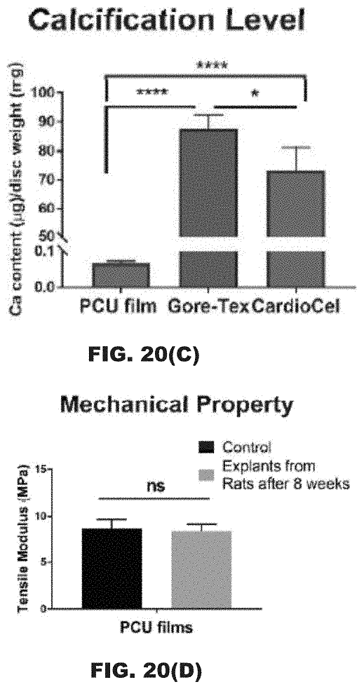

In vivo studies: subcutaneous implantation. The rat subcutaneous implant model was used for screening cellular infiltration, inflammation and calcification resistance in vivo. FIGS. 20(A1)-20(A9) illustrates a set of H&E staining images from PCU film and two commercial patches. Transparent PCU film had delaminated with the adjacent neo-tissue during cutting, due to its elastic properties in comparison with the surrounding tissue. Both the PCU and Gore-Tex samples had a dense layer of tissue capsuled around the samples, and both samples kept a relatively intact morphology after 8-weeks of implantation (FIGS. 20(A1)-20(A3), FIGS. 20(A4)-20(A6)). There was no cell or tissue growth into the PCU film, whereas cellular nuclei were found infiltrated into the Gore-Tex® and CardioCel® patches (FIGS. 20(A5) and 20(A6), FIGS. 20(A8) and 20(A9)). CardioCel® displayed a different tissue response: the patch displayed signs of early degradation and loss of structural integrity at 8 weeks after implantation.

FIGS. 20(A1)-(A9) and 20(B1)-(B6) illustrate histological characterization, mechanical property and calcium quantification of the PCU film, Gore-Tex® patch and CardioCel® Patch after in vivo implantation. Histological sections of the PCU film (FIGS. 20(A1)-20(A3), 20(B1) and 20(B2)), Gore-Tex® (FIGS. 20(A4)-20(A6), 20(B3) and 20(B4)) and CardioCel® (FIGS. 20(A7)-20(A9), 29(B5) and 20(B6)) samples following an 8-weeks subcutaneous implantation in rats. Hematoxylin-eosin staining FIG. 20(A1)-20(A9), alizarin red staining FIG. 20 (B1)-20(B6) The PCU specimen (dash line) had delaminated with the adjacent neo-tissue during microtome cutting due to the elastic properties in comparison with the surrounding tissue.

Sections of the three samples also displayed signs of calcification (red and auburn color) in materials and their surrounding tissues (FIG. 20(B1)-20(B6)). PCU film had no evidence of calcification as indicated in FIGS. 20(B1)-20(B2). Additionally, alizarin red staining showed a far higher degree of calcification in the two commercial patches. The calcification also appeared to extend into surrounding tissues, especially at the interface between the tissue and the two commercial samples (FIGS. 29(B3)-20(B6)). Little to no calcification was present in the rest of the encapsulated tissue.

A calcium content assay was subsequently conducted to confirm the histological findings. A significant increase in Ca²⁺ level was found in Gore-Tex® and CardioCel® samples compared to the PCU film (FIG. 20(C), Table 9). There was no significant difference in tensile modulus of the PCU film before and after implantation, indicating an intact and non-degradable structure of the raw material of the BMM (FIG. 20(D)).

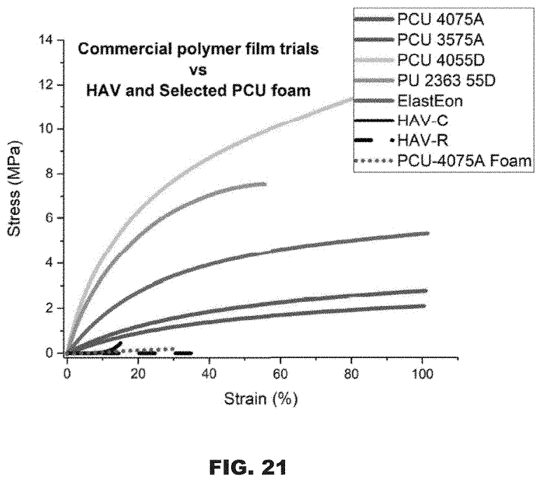

Heart valve leaflets have a highly organized architecture with three specific layers. The fibrosa and ventricularis consist of circumferentially oriented collagen fibers and radially oriented elastin sheets, which constitute their primary load-bearing properties. The spongiosa is inherently soft and compliant with a much lower stiffness. It acts as a cushion, absorbing the load resulting in minimal stress. In this present work, we designed and fabricated a biomimetic, multilayered material to replicate the architecture of those specific layers: The fiber-enhanced PCU films are used as F-mimic layer and V-mimic layers to provide the appropriate mechanical strength and anisotropic properties. A PCU-foam was fabricated via a lyophilization process to create the porous structure from the same polymer solution. It was applied to replicate the load-bearing mechanical role, confer flexibility and tune the overall mechanical properties of BMM. PCU is known as a biostable and biocompatible polymer for heart valve and vascular graft applications. It was found to have superior resistance to degradation under biological conditions when compared with common poly(ether urethane) (PEU) and poly(ether urethane urea) (PEUU). The selection of Carbothane™ AC-4075A as our PCU resin was not only because of its biostable nature, but also due to its mechanical properties in the range of the native tissue (FIG. 21). In order to offer anisotropic behavior and increase the mechanical stiffness in specific directions, aligned PCL fibers are used since electrospun PCL fibers are widely applied in the fabrication of biomedical devices. Utilizing PCU as the main component and PCL as supporting fibers, we fabricated the BMM and assessed its mechanical and biological performance in vitro and in vivo.

The mechanical assessment utilized cyclic uni-axial tensile tests, flexural bulge tests, and suture retention tests for characterization. For the tensile test, our averaged stress-strain curves and modulus data displayed that anisotropic behavior and mechanical properties of native HAVs were not achieved by the commercial patches. Compared to the native tissue, three selected commercial patches are either too stiff or isotropic and are therefore far from a satisfactory material to match the native tissue: Gore-Tex® is the expanded polytetrafluoroethylene (e-PTFE) made through a thermal extrusion and it has the most homogeneous performance (e.g. isotropic) among the three commercial patches. It is also the stiffest sample since the carbon atoms in the ePTFE chain are enclosed within a sheath of fluorine atoms. CorMatrix® and CardioCel® are two tissue-derived products: The former is composed of porcine small intestinal submucosa extracellular matrix and the latter is a tissue-engineered ADAPT bovine pericardial patch. Both of them are less stiff due to the tissue nature, and CardioCel® even has a similar tensile modulus to those of HAV in the C-direction. BMM, in comparison, demonstrated a superior, stable performance with valve-mimicking architecture, anisotropic behavior, and stable tensile modulus. The capability of BMM to match the mechanical performance of the native tissue is important to optimize leaflet stresses and decrease tears and perforations. Mismatched properties, especially high stiffness from a rigid material, will lead to fibrosis, inflammation, and loss of elasticity and functionality.

For the flexural properties, bulge tests were first introduced to study the native leaflet tissue and its artificial alternatives in the literature, to the best of our knowledge. The three commercial patches generally displayed either isotropic or uncontrolled and variable anisotropic performance, and they possessed much higher flexural modulus than HAVs. Both BMM and HAV have a lower flexural modulus and this performance is also in line with the trend of tensile modulus data, especially in the C-direction (FIG. 16, orange line vs red bar). BMM, therefore offers a flexural modulus closer to the native leaflets, compared to the three commercial materials.

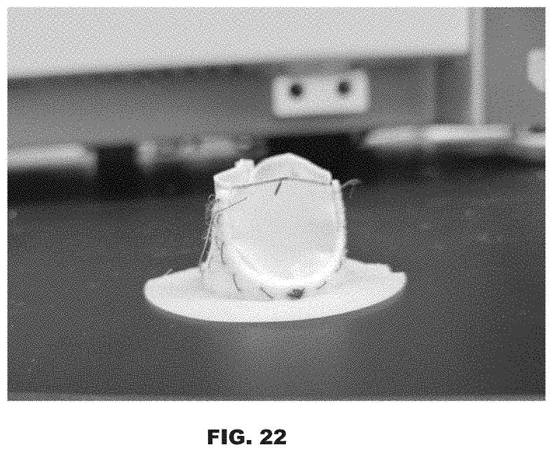

Punctures and defects are generated during suturing the artificial materials, leading to mechanical failures through crack propagation. The resistance to tearing is therefore essential to evaluate the feasibility of patches or alternative materials. The SRS and TN-SRS measurements exhibited that the BMMs have a comparable tear resistance to the commercial products. The BMM also displayed a higher toughness (Table 8) than most commercial patches, which emphasizes its durability and capacity to withstand more tear energy than other samples during suturing. A customized heart valve prototype is also fabricated via suturing the BMMs to the 3D-printed valve struts (FIG. 22) and this prosthesis has been tested via the pulse duplicator and more data about its hydrodynamic performance will be reported in our future work.

The biological assessment of the BMMs and commercial patches included their biostability and biocompatibility, in vitro and in vivo. PCU was selected due to its expected stable and compatible in vivo profile and its stable mechanical properties over time. The degradation of polyurethane-based materials in vitro and in vivo was attributed to several mechanisms, including metal ion-induced accelerated oxidative degradation, hydrolytic degradation and enzymatic degradation. It has been demonstrated that oxidative degradation is the more dominant mechanism over others. Consequently, a 0.1 M CoCl₂/20% H₂O₂ solution was applied to accelerate oxidative degradation of the PCUs. Degradation results after 24 days is shown to correlate to 12 months of in vivo implantation. The modulus of the BMM and PCU film/foam displayed no significant change in mechanical properties for 30 days in this accelerated oxidization solution. It demonstrated that the BMM has a stable performance which is equivalent to 15 months of in vivo implantation. Using polycarbonate macrodiols as the soft segments, the PCU is designed with better hydrolytic stability and anti-oxidization capability than PEU and PEUU. A stable mechanical performance is essential to maintain the mechanical functionality of the valve or patch over time, and to avoid potential failure and repeated reimplantation procedures. These results confirm the biostability of the BMM, which is comparable to the biostable FDA-approved patch (Gore-tex®). However, it is also noted that minor signs of oxidative degradation (FIG. 19(C)) were found on the surface layer, suggesting potential susceptibility of the BMM to long-term oxidization starting from the surface. The biostability of the BMM needs to be assessed in long-term in vivo experiment and may need to be further improved through a surface modification process targeting resistance to oxidation.

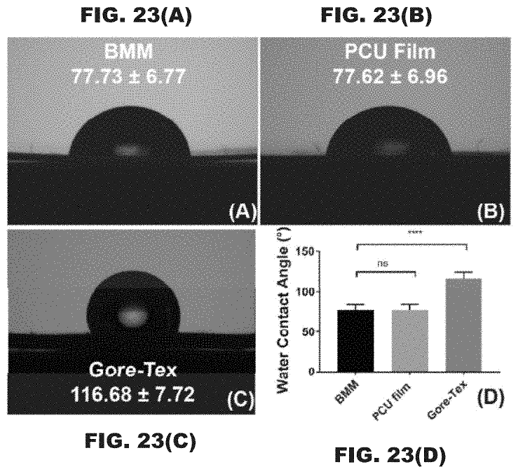

Serum protein adsorption and calcium deposition were examined to evaluate the samples' biocompatibility. Protein adsorption is a significant factor to determine the thrombogenicity of an implanted material. When blood gets in contact with the material's surface, protein adsorption occurs first, which can then provoke the adhesion of platelets and immune cells on the protein layer. Platelets may aggregate continuously and eventually lead to the generation of a non-soluble fibrin network and thrombus formation. An ideal valve leaflet material should have a low protein adsorption profile to limit or cut the path of thrombin formation and potential subsequent thrombogenic reactions. We performed a BSA adsorption test and found that the BMM exhibited a lower level of protein adsorption compared to three commercial patches. Although the difference is not significant, BMM (main composition PCU) displayed a lower surface tension with improved hydrophilicity (FIGS. 23(A)-(D)) compared to Gore-Tex®, which reduces protein adsorption. Its smooth, non-permeable surface is another aspect that limits albumin adsorption. It is also significant to evaluate the resistance to calcification when developing any biomaterial for heart valve application since calcification is the leading reason of failure of bioprosthetic heart valves and grafts. It is a complex phenomenon influenced by a series of mechanical and biochemical factors. Calcification limits the durability of synthetic polymer materials used in heart valve devices and blood contact application in general. A 16-day test showed that the BMM and its main component PCU film had a lower level of Ca2+ ion accumulation compared to commercial patches. This trend is also in line with the findings from in vivo subcutaneous model (FIG. 20(C)). The BMM displayed a higher mean value than the pristine film, which may be attributed to its porous S-mimic layer embedded between films offering more areas for Ca2+ ion accumulation. The BMM should therefore be expected to have a slightly higher calcification level than pristine PCU film, but much lower than commercial patches.

The implantation of any artificial material inevitably provokes a host response. The formation of encapsulated tissue (stained as the pink color in FIGS. 20(A1)-20(A9)) indicates the end stage of a foreign body reaction. The fibrous tissue capsules around PCU film and Gore-Tex® were more organized and denser than that around CardioCel®. The presence of infiltrated cells in Gore-Tex® and CardioCel® samples may be associated with the formation of calcium deposits, and the findings in FIGS. 20(B1)-(B6) supported this hypothesis, as calcium concentrations in these two commercial patches were significantly higher than the value for PCU film. Polymers have relatively superior resistance to calcification compared to tissue-derived materials due to their lack of mineralization, which interacts with phosphorus-rich cellular debris and destroyed collagen. In our comparison of two polymer samples, PCU film had a superior resistance to calcification than Gore-Tex from the in vitro and in vivo quantitative analysis. This indicates that PCU may be option for the development of polymeric heart valve devices including patches and heart valve prostheses.

Compared to three commercial patches, the BMM of the disclosed subject matter demonstrated an anisotropic mechanical behavior and mechanical stiffness which was much closer to the native aortic valve leaflets than the commercial patches. This BMM also showed an excellent durability in an in vitro accelerated oxidization solution and displayed excellent biocompatibility with a lower in vitro protein adsorption level and a lower calcium deposition level. In vivo rat subcutaneous modeling confirmed the mechanical biostability and superior resistance to inflammation and calcification of the main component material, PCU, compared to the commercial patches. This BMM is useful for surgical valve repair and polymeric surgical or transcatheter valve device.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments and/or implementations can be understood and effected by those skilled in the art in practicing the claimed disclosure, from a study of the drawings, the disclosure and the appended claims.

What is claimed is:

1. A biomimetic biomaterial patch configured to mimic native heart valve tissue, the patch comprising a composite body including:
a polymeric Fibrosa-mimic (“F-mimic”) layer;
a polymeric Spongiosa-mimic (“S-mimic”) layer; and
a polymeric Ventricularis-mimic (“V-mimic”) layer.

2. The biomimetic biomaterial patch of claim 1, wherein the F-mimic layer and the V-mimic layer of the composite body are anisotropic and the S-mimic layer is a shock absorbing layer.

3. The biomimetic biomaterial patch of claim 1, wherein the F-mimic layer is formed of polycarbonate polyurethane (PCU) film having embedded polycaprolactone (PCL) fibers therein.

4. The biomimetic biomaterial of claim 1, wherein the V-mimic layer is formed of polycarbonate polyurethane (PCU) film having embedded polycaprolactone (PCL) fibers therein.

5. The biomimetic biomaterial patch of claim 3, wherein at least one of the F-mimic and the V-mimic layers are each made of polycarbonate polyurethane (PCU) film having embedded aligned polycaprolactone (PCL) fibers therein.

6. The biomimetic biomaterial patch of claim 5, wherein the PCL fibers are electrospun.

7. The biomimetic biomaterial patch of claim 6, wherein the electrospun fibers exhibit a highly oriented distribution.

8. The biomimetic biomaterial patch of claim 7, wherein the electrospun fibers exhibit anisotropic performance during cyclic tensile tests compared to random electrospun PCL fibers.

9. The biomimetic biomaterial patch of claim 7, wherein the F-mimic and V-mimic layers demonstrate anisotropic behavior in first and second directions, the first and second directions being different directions.

10. The biomimetic biomaterial patch of claim 9, wherein the first direction is the H direction and the second direction is the V direction.

11. The biomimetic biomaterial patch of claim 5, wherein the tensile modulus are from about 25 to about 40 MPa at the strain in the H direction and about 1 to 3 MPa at the strain in the V direction.

12. The biomimetic biomaterial patch of claim 1, wherein the S-mimic layer is formed from PCU foam.

13. The biomimetic biomaterial patch of claim 1, wherein the F-mimic layer is coated on one surface of the S-mimic layer and the V-mimic layer is coated on the opposing surface of the S-mimic layer to form a composite biomimetic patch structure.

14. The biomimetic biomaterial patch of claim 1, wherein the biomimetic patch exhibits anisotropic mechanical behavior similar to native human valve leaflets.

15. The biomimetic biomaterial patch of claim 1, wherein the biomaterial exhibits a tensile modulus of about 4 to 8 MPa at the strain in a first direction.

16. The biomimetic biomaterial patch of claim 1, wherein the biomaterial exhibits a tensile modulus of about 1.5 to 2 MPa at the strain in a second direction.

17. The biomimetic biomaterial patch of claim 1, wherein the S-mimic layer is made of PCU foam and the F-mimic and the V-mimic layers are each made of polycarbonate polyurethane (PCU) film having embedded electrospun, aligned polycaprolactone (PCL) fibers therein.

18. The biomimetic biomaterial patch of claim 1, wherein the patch is entirely made of polymeric material.

19. The biomimetic biomaterial patch of claim 1, wherein the patch is limited to three layers.

20. The biomimetic biomaterial patch of claim 1, wherein the patch is mechanically stable for clinical use.

21. A stable biomimetic biomaterial comprising
a first layer comprising polycarbonate polyurethane (PCU) film embedded with aligned polycaprolactone (PCL) fibers,
a second layer comprising PCU foam, and
a third layer comprising polycarbonate polyurethane (PCU) film embedded with aligned polycaprolactone (PCL) fibers, wherein the layers form a composite structure.

22. The stable biomimetic biomaterial of claim 21, wherein the composite structure lacks animal-derived tissue.

23. The stable biomimetic biomaterial of claim 21, wherein the patch exhibits a low protein adsorption.

24. The stable biomimetic biomaterial of claim 21, wherein the patch exhibits low Ca²⁺ adhesion.

25. The stable biomimetic biomaterial of claim 21, further comprising a surface layer disposed at least one surface of the composite structure.

26. The stable biomimetic biomaterial of claim 25, wherein the surface layer comprises Parylene C.

27. The stable biomimetic biomaterial of claim 21, at least one surface of the composite structure includes a corrugated structure to mimic morphology of the native heart leaflet surface.

28. The stable biomimetic biomaterial of claim 21, wherein the biomaterial exhibits anisotropic mechanical behavior similar to native human valve leaflets.

29. The stable biomimetic biomaterial of claim 21, wherein the biomaterial exhibits a tensile modulus of about 4 to 8 MPa at the strain in a first direction.

30. The stable biomimetic biomaterial of claim 29, wherein the biomaterial exhibits a tensile modulus of about 1.5 to 2 MPa at the strain in a second direction.

31. The stable biomimetic biomaterial of claim 29, wherein the material is in the form of a heart patch repairing material.

32. An implantable prosthetic heart valve comprising:
a plurality of leaflets, each leaflet formed from a polymeric biomaterial, wherein the polymeric biomaterial is a composite body including a polymeric Spongiosa-mimic (“S-mimic”) layer and at least one polymeric layer selected from the group consisting of: a polymeric Fibrosa-mimic (“F-mimic”) layer; and a polymeric Ventricularis-mimic (“V-mimic”) layer, or a combination thereof.

33. The prosthetic heart valve of claim 32, wherein the F-mimic layer and the V-mimic layer are anisotropic and the S-mimic layer is a shock absorbing layer each leaflet.

34. The prosthetic heart valve of claim 32, wherein the F-mimic layer is formed of polycarbonate polyurethane (PCU) film having embedded polycaprolactone (PCL) fibers therein.

35. The prosthetic heart valve of claim 34, wherein the embedded PCL fibers are aligned fibers.

36. The prosthetic heart valve of claim 34, wherein the PCL fibers are from electrospun.

37. The prosthetic heart valve of claim 32, wherein the prosthetic comprises a tri-layered composite body including:
a polymeric Fibrosa-mimic (“F-mimic”) layer;
a polymeric Spongiosa-mimic (“S-mimic”) layer; and
a polymeric Ventricularis-mimic (“V-mimic”) layer.

38. The prosthetic heart valve of claim 37, wherein at least one of the F-mimic and the V-mimic layers are each made of polycarbonate polyurethane (PCU) film having embedded aligned polycaprolactone (PCL) fibers therein.

39. The prosthetic heart valve of claim 38, wherein the PCL fibers are electrospun.

40. The prosthetic heart valve of claim 39, wherein the electrospun fibers exhibit a highly oriented distribution.

41. The prosthetic heart valve of claim 39, wherein the electrospun fibers exhibit anisotropic performance during cyclic tensile tests compared to random electrospun PCL fibers.

42. The prosthetic heart valve of claim 41, wherein the F-mimic and V-mimic layers demonstrate anisotropic behavior in first and second directions, the first and second directions being different directions.

43. The prosthetic heart valve of claim 42, wherein the first direction is the H direction and the second direction is the V direction.

44. The prosthetic heart valve of claim 43, wherein the tensile modulus are from about 25 to about 40 MPa at the strain in the H direction and about 1 to 3 MPa at the strain in the V direction.

45. The prosthetic heart valve of claim 37, wherein the S-mimic layer is formed from PCU foam.

46. The prosthetic heart valve of claim 37, wherein the F-mimic layer is coated on one surface of the S-mimic layer and the V-mimic layer is coated on the opposing surface of the S-mimic layer to form a composite biomimetic patch structure.

47. The prosthetic heart valve of claim 37, wherein the composite body exhibits anisotropic mechanical behavior similar to native human valve leaflets.

48. The prosthetic heart valve of claim 37, wherein the composite body exhibits a tensile modulus of about 4 to 8 MPa at the strain in a first direction.

49. The prosthetic heart valve of claim 37, wherein the composite body exhibits a tensile modulus of about 1.5 to 2 MPa at the strain in a second direction.

50. The prosthetic heart valve of claim 36, wherein the S-mimic layer is made of PCU foam and the F-mimic and the V-mimic layers are each made of polycarbonate polyurethane (PCU) film having embedded electrospun, aligned polycaprolactone (PCL) fibers therein.

51. The prosthetic heart valve of claim 36, wherein the composite body is entirely made of polymeric material.

52. The prosthetic heart valve of claim 36, wherein the composite body includes from two to five polymeric layers.

53. The prosthetic heart valve of claim 36, wherein the composite body lacks animal-derived tissue.

54. The prosthetic heart valve of claim 36, wherein the prosthetic heart valve is an aortic valve, mitral valve, or a tricuspid valve.

55. A method of treating a heart defect comprising the steps of:
providing a biomimetic biomaterial patch according to claim 1,
placing the biomimetic biomaterial patch on an uninflated distal balloon,
placing the biomimetic biomaterial patch and the balloon distally of the defective opening;
inflating the balloon,
moving the balloon and the patch on the balloon firmly against the defective opening,
permitting the patch to contact to the heart defect, then deflating and removing the balloon.

56. The method of claim 55, wherein the biomimetic biomaterial patch endothelializes to the defect.

57. The method of claim 55, wherein the biomimetic biomaterial patch occludes the heart defect.

58. The method of claim 55, wherein the biomimetic biomaterial patch is percutaneously delivered to the heart defect.

59. The method of claim 55, wherein the balloon is mounted on a delivery catheter.

60. The method of claim 55, wherein the biomimetic biomaterial patch has a shape that matches the shape of the cardiac site to be repaired.

61. A method of replacing a heart valve in a subject, comprising the steps of:
inserting a distal end portion of a delivery sheath into a portion of a heart of a subject, the delivery sheath having a prosthetic heart valve according to claim 32 disposed within a lumen of the delivery sheath,
moving the prosthetic heart valve distally out of the delivery sheath; and
positioning the prosthetic heart valve within the heart.

62. The method of claim 61, wherein the method is a method is a method for treating the subject for aortic stenosis, mitral valve stenosis, regurgitation, or tricuspid valve regurgitation.

63. A stable biomimetic biomaterial comprising
a polycarbonate polyurethane (PCU) foam layer, and
a plurality of aligned polypropylene fibers embedded in the PCU layer, such that the plurality of aligned polypropylene fibers are spaced from each other.

64. The stable biomimetic biomaterial of claim 63, wherein the polypropylene fibers are sutures.

65. The stable biomimetic biomaterial of claim 63, wherein the plurality of propylene fibers includes fibers having different sizes.

66. The stable biomimetic biomaterial of claim 65, wherein the sizes of the polypropylene fibers are selected from the group consisting of 6-0, 7-0, and 8-0.

67. The stable biomimetic biomaterial of claim 63, wherein the plurality of polypropylene fibers includes up to 4 fibers.

68. The stable biomimetic biomaterial of claim 63, wherein the biomaterial exhibits a tensile modulus in a C/H direction of about 8 to about 16 MPa.

69. The stable biomimetic biomaterial of claim 63, wherein the biomaterial exhibits a tensile modulus in a R/V direction of about 0.57 to about 0.68 MPa.
